SEQUENCE LISTING (41 sequences)

SEQ ID NO: 1 - DNA, 975 bp, Hibiscus cannabinus
atggtgttattatcaaaacctggaattgaacagttttcttatgttagaaacaagcagccagccgcattgttccctcctcttatccctatcgtagacctctccgaaccggattccagacaccagatgatcaaagcctgcgaggaattcggattcttcaaggtgatcaaccatggggttcccatggaattcatttccaggcttgaatccgaggccacccagttcttctctttaccgctttcccagaaagagaaaacagggcagcccaaaccttacggatatggcaatagaaggatcggtcccaatggtgatgttggttggctcgaatatctcctcctcacgaccaaccaagaccccaacctcccaactgaaaacccagatggtttcagggtagctttgaataattatatggcggcggtgaagaaactggcgtgcgagatacttgaaatggtggcggatgggctaaagattcaacctcggaatgtgttgagtaagctgatgatggatgagcagagtgactcggttttcaggctgaaccattaccctccatgcccatcgtcgaatggaaatgtgattggattcggcgaacacactgacccacaaatcatttcggtcctaagatccaacaacacttctggccttcaaatctctctaagagacggctcttggatttcagtcccacccgaccaccactcatttttcatcaatgttggtgattccttacaggtaatgacgaatgggaggttcaaaagtgtgaagcatagggtgttggcgaacagtgtgaaatcaaggctgtcaatgatatatttctgtggaccgccaatgagtgagaagatagccccattgccatgtttgatgaggggagatcagcaaagcttgtacaaagaatttacatggtttgactacaaaacttctgcttacaactctaggttggcagataacaggctaatcaacttcgagaaaatagctgcctcataa

SEQ ID NO: 2 - protein, 324 aa, Hibiscus cannabinus
MVLLSKPGIEQFSYVRNKQPAALFPPLIPIVDLSEPDSRHQMIKACEEFGFFKVINHGVPMEFISRLESEATQFFSLPLSQKEKTGQPKPYGYGNRRIGPNGDVGWLEYLLLTTNQDPNLPTENPDGFRVALNNYMAAVKKLACEILEMVADGLKIQPRNVLSKLMMDEQSDSVFRLNHYPPCPSSNGNVIGFGEHTDPQIISVLRSNNTSGLQISLRDGSWISVPPDHHSFFINVGDSLQVMTNGRFKSVKHRVLANSVKSRLSMIYFCGPPMSEKIAPLPCLMRGDQQSLYKEFTWFDYKTSAYNSRLADNRLINFEKIAAS

SEQ ID NO: 3 - DNA, 1134 bp, Hibiscus cannabinus
atggtgttattatcaaaacctggaattgaacagttttcttatgttagaaacaagcagccagccgcattgttccctcctcttatccctatcgtagacctctccgaaccggattccagacaccagatgatcaaagcctgcgaggaattcggattcttcaaggtgatcaaccatggggttcccatggaattcatttccaggcttgaatccgaggccacccagttcttctctttaccgctttcccagaaagagaaaacagggcagcccaaaccttacggatatggcaatagaaggatcggtcccaatggtgatgttggttggctcgaatatctcctcctcacgaccaaccaagaccccaacctcccaactgaaaacccagatggtttcaggtaccctttttttttccttcttaccagagatagggaatctgttatcacagcttttgaaactgtgtctgtgttgttatttttcagggtagctttgaataattatatggcggcggtgaagaaactggcgtgcgagatacttgaaatggtggcggatgggctaaagattcaacctcggaatgtgttgagtaagctgatgatggatgagcagagtgactcggttttcaggctgaaccattaccctccatgcccatcgtcgaatggaaatgtgattggattcggcgaacacactgacccacaaatcatttcggtcctaagatccaacaacacttctggccttcaaatctctctaagagacggctcttggatttcagtcccacccgaccaccactcatttttcatcaatgttggtgattccttacaggtaaataataaaaaaaattccttcttttttaccaactgtgggtctggtgcaaagtgtgagcttgatgttgttcaggtaatgacgaatgggaggttcaaaagtgtgaagcatagggtgttggcgaacagtgtgaaatcaaggctgtcaatgatatatttctgtggaccgccaatgagtgagaagatagccccattgccatgtttgatgaggggagatcagcaaagcttgtacaaagaatttacatggtttgactacaaaacttctgcttacaactctaggttggcagataacaggctaatcaacttcgagaaaatagctgcctcataa

SEQ ID NO: 4 - DNA, 473 bp, Artificial sequence
  Synthetic polynucleotide
gtgaggagtttggtttcttcaaagtgatcaaccatggggttcccatggaattcatttccaggcttgaatctgaagccaccgagttcttctctttgccgctttctgagaaggagaaaacagggcagcccaaaccttatggatatggcaataaaaggatcggtcccaatggtgatgttggttgggttgaatatcttctcctcatgaccaaccaagaccccaacctcgctactgaaaacccagaaagtttcagggttgctttgaataattatatggcggcggtgaagaagatggcgtgcgagatactggaaatggtggcggatgggctgaagattcaaccgaggaatgtgttgagcaagctgatgatggacgagcagagtgactctgttttcaggctgaaccattaccctccatgcccagatgttgttcaatctctcaacgacaatgtgattggatttggggagcacacagaccca

SEQ ID NO: 5 - DNA, 22 bp, Artificial sequence
  Synthetic Oligonucleotide
caagacccttcctctatataag

SEQ ID NO: 6 - DNA, 20 bp, Artificial sequence
  Synthetic Oligonucleotide
ttatcctagtttgcgcgcta

SEQ ID NO: 7 - DNA, 18 bp, Artificial sequence
  Synthetic Oligonucleotide
tgtaaaacgacggccagt

SEQ ID NO: 8 - DNA, 22 bp, Artificial sequence
  Synthetic Oligonucleotide
aacagctatgaccatgattacg

SEQ ID NO: 9 - DNA, 19 bp, Artificial sequence
  Synthetic Oligonucleotide
gctcctacaaatgccatca

SEQ ID NO: 10 - DNA, 20 bp, Artificial sequence
  Synthetic Oligonucleotide
gatagtgggattgtgcgtca

SEQ ID NO: 11 - DNA, 32 bp, Artificial sequence
  Synthetic Oligonucleotide
atggatcctatggccgtaagtttcgtaacaac

SEQ ID NO: 12 - DNA, 36 bp, Artificial sequence
  Synthetic Oligonucleotide
acgaattcggttagatgggtttggtgagccaatctg

SEQ ID NO: 13 - DNA, 31 bp, Artificial sequence
  Synthetic Oligonucleotide
ggtctagaatggccgtaagtttcgtaacaac

SEQ ID NO: 14 - DNA, 25 bp, Artificial sequence
  Synthetic Oligonucleotide
cctctagagatgggtttggtgagcc

SEQ ID NO: 15 - DNA, 28 bp, Artificial sequence
  Synthetic Oligonucleotide
cctctagattagatgggtttggtgagcc

SEQ ID NO: 16 - DNA, 36 bp, Artificial sequence
  Synthetic Oligonucleotide
ccatcgatggtacccctgaaagttcccgggcttttg

SEQ ID NO: 17 - DNA, 40 bp, Artificial sequence
  Synthetic Oligonucleotide
cgggatcccggaattccgcaatggcggtattgtctaaacc

SEQ ID NO: 18 - DNA, 25 bp, Artificial sequence
  Synthetic Oligonucleotide
actcgagagattggatttggtgagc

SEQ ID NO: 19 - DNA, 25 bp, Artificial sequence
  Synthetic Oligonucleotide
aatcgataacctgcaatgagtcacc

SEQ ID NO: 20 - DNA, 25 bp, Artificial sequence
  Synthetic Oligonucleotide
aggtaccaacctgcaatgagtcacc

SEQ ID NO: 21 - DNA, 25 bp, Artificial sequence
  Synthetic Oligonucleotide
gtctagaagattggatttggtgagc

SEQ ID NO: 22 - DNA, 28 bp, Artificial sequence
  Synthetic Oligonucleotide
gatcgatgcagagtcactctgctcgtcc

SEQ ID NO: 23 - DNA, 28 bp, Artificial sequence
  Synthetic Oligonucleotide
cggtaccccagagtcactctgctcgtcc

SEQ ID NO: 24 - DNA, 29 bp, Artificial Synthetic
  Synthetic Oligonucleotide
gtctagaggtgatcaaccatggggttccc

SEQ ID NO: 25 - DNA, 29 bp, Artificial sequence
  Synthetic Oligonucleotide
cctcgagggtgatcaaccatggggttccc

SEQ ID NO: 26 - DNA, 20 bp, Artificial sequence
  Synthetic Oligonucleotide
gggcagcccaaaccttatgg

SEQ ID NO: 27 - DNA, 28 bp, Artificial sequence
  Synthetic Oligonucleotide
atggtgttattatcaaaacctggaattg

SEQ ID NO: 28 - DNA, 30 bp, Artificial sequence
  Synthetic Oligonucleotide
gcctaaaagatagagagattatgaggcagc

SEQ ID NO: 29 - DNA, 25 bp, Artificial sequence
  Synthetic Oligonucleotide
ccatgggaaccccatggttgatcac

SEQ ID NO: 30 - DNA, 25 bp, Artificial sequence
  Synthetic Oligonucleotide
gcggcaaagagaagaactcggtggc

SEQ ID NO: 31 - DNA, 27 bp, Artificial sequence
  Synthetic Oligonucleotide
caggctgaaccattaccctccatgccc

SEQ ID NO: 32 - DNA, 26 bp, Artificial sequence
  Synthetic Oligonucleotide
cgacaatgtgattggatttggggagc

SEQ ID NO: 33 - DNA, 25 bp, Artificial sequence
  Synthetic Oligonucleotide
tgnganganttnggnttnttnaang

SEQ ID NO: 34 - DNA, 23 bp, Artificial sequence
  Synthetic Oligonucleotide
ggntcdgtntgntcncgnaancc

SEQ ID NO: 35 - DNA, 373 bp, Artificial sequence
  Synthetic Polynucleotide
caatggcggtattgtctaaaccggtagcaataccaaaatccgggttctctctaatcccggttatagatatgtctgacccagaatccaaacatgccctcgtgaaagcatgcgaagacttcggcttcttcaaggtgatcaaccatggcgtttccgcagagctagtctctgttttagaacacgagaccgtcgatttcttctcgttgcccaagtcagagaaaacccaagtcgcaggttatcccttcggatacgggaacagtaagattggtcggaatggtgacgtgggttgggttgagtacttgttgatgaacgctaatcatgattccggttcgggtccactatttccaagtcttctcaaaagcccgggaactttcag

SEQ ID NO: 36 - DNA, 155 bp, Artificial sequence
  Synthetic Polynucleotide
gattggatttggagagcatactgacccacaaatcatatcagtattgagatccaacaacacttccggacttcaaatattactcaaaaatggccactggatttctgtcccacctgatccaaattctttcttcattaatgttggtgattcattgcagg

SEQ ID NO: 37 - DNA, 488 bp, Artificial sequence
  Synthetic Polynucleotide
ttgcgaggaatttggtttttttaaagtcgtaaaccatgacgttcctatagaattcataagtaaactcgaatccgaagccatcaaattcttctcctctcccctctctgagaagctaaaagcagggcctgctgacccttttggctatggcaataaacaaatcggacaaagtggcgatatcggttgggttgaatacattctcctttcaacaaactctgaattcaattaccagaaattcgcatctgtattaggtgtcaatccagaaaacattcggtaagtcattaaaagcagagcaaaacagagtgatgtcttttaatctcgattcttgatttttgttattgttattgntattgcagagctgcggngaangattatgtggcagcagtgaagagaangtcatgtgagattcttgaagaagttggcggagggattaaagattcatccgncgaatgttttcggtaagcttttgaaggatgaaaagagcgactctg

SEQ ID NO: 38 - DNA, 473 bp, Artificial sequence
  Synthetic Polynucleotide
tgggtctgtgtgctccccaaatccaatcacattgtcgttgagagattgaacaacatctgggcatggagggtaatggttcagcctgaaaacagagtcactctgctcgtccatcatcagcttgctcaacacattcctcggttgaatcttcagcccatccgccaccatttccagtatctcgcacgccatcttcttcaccgccgccatataattattcaaagcaaccctgaaactttctgggttttcagtagcgaggttggggtcttggttggtcatgaggagaagatattcaacccaaccaacatcaccattgggaccgatccttttattgccatatccataaggtttgggctgccctgttttctccttctcagaaagcggcaaagagaagaactcggtggcttcagattcaagcctggaaatgaattccatgggaaccccatggttgatcactttgaagaaaccaaactcctcac

SEQ ID NO: 39 - DNA, 373 bp, Artificial sequence
  Synthetic Polynucleotide
ctgaaagttcccgggcttttgagaagacttggaaatagtggacccgaaccggaatcatgattagcgttcatcaacaagtactcaacccaacccacgtcaccattccgaccaatcttactgttcccgtatccgaagggataacctgcgacttgggttttctctgacttgggcaacgagaagaaatcgacggtctcgtgttctaaaacagagactagctctgcggaaacgccatggttgatcaccttgaagaagccgaagtcttcgcatgctttcacgagggcatgtttggattctgggtcagacatatctataaccgggattagagagaacccggattttggtattgctaccggtttagacaataccgccattg

SEQ ID NO: 40 - DNA, 155 bp, Artificial sequence
  Synthetic Polynucleotide
cctgcaatgaatcaccaacattaatgaagaaagaatttggatcaggtgggacagaaatccagtggccatttttgagtaatatttgaagtccggaagtgttgttggatctcaatactgatatgatttgtgggtcagtatgctctccaaatccaatc

SEQ ID NO: 41 - DNA, 488 bp, Artificial sequence
  Synthetic Polynucleotide
cagagtcgctcttttcatccttcaaaagcttaccgaaaacattcgncggatgaatctttaatccctccgccaacttcttcaagaatctcacatgacnttctcttcactgctgccacataatcnttcnccgcagctctgcaatancaataacaataacaaaaatcaagaatcgagattaaaagacatcactctgttttgctctgcttttaatgacttaccgaatgttttctggattgacacctaatacagatgcgaatttctggtaattgaattcagagtttgttgaaaggagaatgtattcaacccaaccgatatcgccactttgtccgatttgtttattgccatagccaaaagggtcagcaggccctgcttttagcttctcagagaggggagaggagaagaatttgatggcttcggattcgagtttacttatgaattctataggaacgtcatggtttacgactttaaaaaaaccaaattcctcgcaa

(19) United States
(12) Patent Application Publication
(10) Pub. No.: US 2011/0004958 A1
(43) Pub. Date: Jan. 6, 2011

(54) COMPOSITIONS FOR SILENCING THE EXPRESSION OF GIBBERELLIN 2-OXIDASE AND USES THEREOF

(75) Inventors: Roni Aloni, Tel-Aviv (IL); Adi Avni, Binyamina (IL); Jonathan Dayan, Tel-Aviv (IL)

Correspondence Address: WINSTON & STRAWN LLP;PATENT DEPARTMENT, 1700 K STREET, N.W., WASHINGTON, DC, 20006, US

(73) Assignee: RAMOT AT TEL-AVIV UNIVERSITY LTD

(21) Appl. No.: 12/301,607

(22) PCT Filed: May 24, 2007
(86) PCT No.: PCT/IL2007/000629

§ 371 Date: Sep. 14, 2009

Related U.S. Application Data
(60) Provisional application No. 60/802,516, filed on May 23, 2006.

(51) Int. Cl. A01H 1/00; C07H 21/02; C12N 5/10; A01H 5/00; C07H 21/04

(52) U.S. Cl. 800/285; 536/24.5; 435/419; 800/298; 536/23.2

(57) ABSTRACT

The present invention provides an isolated nucleic acid sequence encoding gibberellin 2-oxidase enzyme (GA 2-oxidase) of Hibiscus cannabinus. Furthermore, the present invention provides methods of silencing the expression of GA 2-oxidase gene via RNA interference for increasing fibers in plants, in particular in kenaf.

### FIELD OF THE INVENTION

The present invention relates to an isolated nucleic acid sequence encoding gibberellin 2-oxidase enzyme (GA 2-oxidase) of Hibiscus cannabinus. The present invention further relates to methods of silencing the expression of GA 2-oxidase gene useful for increasing fibers in plants.

### BACKGROUND OF THE INVENTION

Plant fibers are elongated cells with tapering ends and very thick, heavily lignified cell walls. Many economically important products such as paper, cordage (cords and ropes), and textiles are derived from plant fibers. Fiber cells function as support tissue in plant stems and roots. Fibers are components of sclerenchyma tissue, which also contains the shorter, thick-walled sclereids (stone cells). The fibers are regularly found in the xylem (wood) and phloem (bark) tissues of monocot and dicot stems and roots.

Fibers present a great economical target being among the main commercial products of trees. In 1997, it was predicted that total global consumption of papermaking fiber would increase from about 300 million tons at 1998 to about 425 million tons by the year 2010.

The induction of fiber formation is controlled by the plant hormones auxin and gibberellin produced in leaves, and by cytokinine produced in roots. Auxin and gibberellin also control the formation and structure of lignin in the fiber cell walls. It has been shown that gibberellin is the specific signal that induces fibers in both the xylem and phloem (Aloni, Plant Physiol. 63: 609-614, 1979; Aloni et al., Plant Physiol. 94: 1743-1747, 1990).

Exogenous applications of plant hormones partially overcome the obstacle of slow growth of trees due to different plant molecular regulations.

Gibberellins (GAs) are a group of more than 100 tetracyclic diterpenes, some of which are essential endogenous regulators that influence growth and development processes throughout the plant life cycle, including shoot elongation, the expansion and shape of leaves, flowering, and seed germination. The importance of GAs in the control of shoot elongation is illustrated by GA-deficient mutants of Arabidopsis, maize, and pea. These mutants have reduced levels of active GA(s) compared to wild-type (wt) plants and shorter internodes which result in a dwarf phenotype. The phenotype of such GA-deficient mutants can be completely restored by application of an active GA. Biosynthesis of GAs in plants occurs through the isoprenoid pathway from mevalonic acid. Gibberellin levels are mainly regulated by transcriptional control of gibberellin biosynthesis genes.

The multifunctional enzyme GA 20-oxidase is a key enzyme in controlling GA biosynthesis. It catalyzes the stepwise conversion of the C20 gibberellins, GA12/GA53, by three successive oxidations to GA9/GA20, which are the immediate precursors of the bioactive gibberellins, GA4 and GA1, respectively. The expression of the GA 20-oxidase gene is down regulated by the action of GA1/4, suggesting that direct end-product repression is involved in the regulation of the gene. The first step of degradation of biological active GAs involves GA 2-oxidases that hydroxylate the C-2 of active GAs. GA 2-oxidase genes have been cloned from several species (Thomas et al. Proc. Natl. Acad. Sci. U.S.A., 96 (1999) 4698-4703).

A slender mutant having high levels of GAs was shown to exhibit hyperelongation. The SLENDER gene encoding a GA 2-oxidase revealed the role of GA 2-oxidases in controlling GAs levels. Indeed, over-expression of GA 2-oxidase in Arabidopsis caused reduction in GA levels resulting in dwarf phenotypes typical of mutants with GA deficiency.

To date, most of the genetic research concerning fiber yield enhancement has been focused on over-expression of GA-20 oxidase. In transgenic plants which over-express GA-20 oxidase, high levels of GAs in both leaves and internodes were measured, demonstrating that GA biosynthesis was enhanced. These transgenic plants show increased fiber length (Eriksson et al. Planta, 214: 920-930, 2002; Huang et al., Plant Physiol. 118: 773-781, 1998; and Biemelt et al., Plant Physiol. 135: 254-265, 2004), and extremely high levels of inactive GAs, due to GA 2-oxidase catalysis.

U.S. Pat. No. 6,670,527 to Thomas et al. discloses nucleic acid sequences encoding GA-2 oxidase enzymes in Phaseolus coccineus and Arabidopsis thaliana and amino acid sequences of these GA 2-oxidases. U.S. Pat. No. 6,670,527 is directed to methods of inhibiting plant growth comprising transforming a plant cell with a nucleic acid which expresses a plant polypeptide having GA 2-oxidase enzyme activity. U.S. Pat. No. 6,670,527 further teaches antisense nucleic acid sequences and nucleic acid sequences encoding a ribozyme that may be useful for promoting plant growth by preventing the deactivation of gibberellin by GA 2-oxidase.

U.S. Pat. No. 6,723,897 to Brown et al. discloses methods for reducing GA levels in plants by producing transgenic plants having a transgene that comprises a sequence that encodes GA 2-oxidase, the latter inactivates an endogenous GA.

U.S. Pat. No. 4,507,144 to Aloni discloses a process of increasing the fiber crop of plants comprising applying to the plants in combination an auxin and gibberellin a number of times, and harvesting the crop after the plants reach the desired stage of growth.

Kenaf (Hibiscus cannabinus) is a fast growing annual plant closely related to cotton (Gossypium hirsutum L.) reaching heights of 4-6 meters within 6 months, and achieving 5-10 tons of dry fiber/acre. It is an environmentally friendly plant which has been used for a long time as a source for the production of cordage and ropes. Studies with kenaf in the United States have been mostly concerned with yield production. While some of the research of the 1970's and 1980's related to the use of kenaf in newsprint, the commercialization of kenaf into various products has become the main focus in recent years. Kenaf is an advantageous fiber resource since it requires minimal maintenance and few or no herbicides due to its quick germination and emergence. The dense canopy of leaves at the top of the plant chokes out weed growth. In addition, kenaf needs about the same amount of fertilizer as upland cotton and less than corn. Most insect pests do not attack the fibrous plant stem, and therefore insecticides are unnecessary.

With concerns of a shortage of softwood to supply increasing demand for pulp and paper products, kenaf is a promising pulp alternative. As kenaf grows quickly, this plant is a better source of fiber than Southern pine trees, currently being used for pulp in the United States, as the time needed to reach harvestable size in Southern pine trees ranges from 7 to 40 years.

There is an unmet need for effective and improved methods for increasing bark (phloem) and wood (xylem) fibers in plants in general and in kenaf plants (Hibiscus cannabinus), in particular.

### SUMMARY OF THE INVENTION

The present invention provides an isolated nucleic acid sequence encoding gibberellin 2-oxidase (GA 2-oxidase) enzyme of Hibiscus cannabinus (kenaf) and an amino acid sequence of the enzyme. The present invention further provides nucleic acid sequences useful for silencing gene expression of GA 2-oxidase in plants and methods of increasing the fiber crop in plants by silencing gene expression of GA 2-oxidase.

It is now disclosed for the first time that transformation of Arabidopsis plants with a DNA construct designed for generating siRNAs targeted to the GA 2-oxidase gene of Arabidopsis plants resulted in the production of transgenic plants in which the GA 2-oxidase gene expression was silenced by RNA interference (RNAi). The transgenic Arabidopsis plants were found to be taller than non-transgenic wild type Arabidopsis plants, their internodes were longer, and they flowered faster than their wild type counterpart plants. In addition, the number of fiber cells in stems of the transgenic Arabidopsis plants was higher than that of the wild type plants.

It is further disclosed that transformation of tobacco plants with a DNA construct designed for generating siRNAs targeted to the GA 2-oxidase gene of tobacco plants resulted in the production of transgenic plants in which the GA 2-oxidase gene expression was silenced. The transgenic tobacco plants were found to be taller than non-transgenic wild type tobacco plants. The leaves of the transgenic tobacco plants were longer than those of the wild type plants.

It is now disclosed that transgenic Arabidopsis and tobacco plants in which GA 2-oxidase is silenced exhibit similar phenotypic characteristics as those of transgenic Arabidopsis and tobacco plants in which GA 20-oxidase is over-expressed. Thus, the present invention surprisingly discloses that increase in gibberellin levels in plants by virtue of silencing the expression of a GA 2-oxidase gene produces faster growing and taller plants which have higher phloem and/or xylem fiber content than non-transgenic wild type plants.

The present invention further discloses a DNA sequence encoding a GA 2-oxidase enzyme of Hibiscus cannabinus (kenaf) as set forth in SEQ ID NO:1, the amino acid sequence of the GA 2-oxidase enzyme of kenaf as set forth in SEQ ID NO:2, and the genomic sequence of the GA 2-oxidase of kenaf as set forth in SEQ ID NO:3. The present invention further discloses DNA sequences capable of generating RNA molecules capable of inhibiting the expression of GA 2-oxidase genes of various plants. The DNA sequences are useful for transforming plants in order to silence the expression of GA 2-oxidase gene so that the level of gibberellin in these plants is reduced and the amount and density of fibers in the transformed plants is higher than that of control plants. Such transformed plants can be a valuable source of pulp and fiber. The DNA sequences of the present invention are particularly useful for transforming kenaf plants.

The present invention is exemplified herein in Arabidopsis and tobacco plants. However, any plant used commercially as a source of fiber can be a better and enriched source of fiber after being transformed with a DNA construct capable of generating siRNAs targeted to the GA 2-oxidase gene of that plant, and is thus within the scope of the present invention.

According to a first aspect, the present invention provides an isolated nucleic acid molecule comprising the nucleotide sequence as set forth in SEQ ID NO:1 encoding a gibberellin 2-oxidase enzyme (GA 2-oxidase) of Hibiscus cannabinus (kenaf), a fragment, a complementary strand or a homologous sequence thereto, wherein the homologous sequence has sequence identity of at least 80% to SEQ ID NO:1. According to some embodiments, the homologous sequence has sequence identity of at least 90%, alternatively of at least 95%, alternatively of 100% to SEQ ID NO:1. According to a certain embodiment, the isolated nucleic acid molecule has the nucleotide sequence as set forth in SEQ ID NO:1 encoding a GA 2-oxidase of kenaf. According to another embodiment, the isolated nucleic comprises the nucleotide as set forth in SEQ ID NO:4.

According to a further aspect, the present invention provides an isolated GA 2-oxidase polypeptide of kenaf (Hibiscus cannabinus) comprising the amino acid sequence as set forth in SEQ ID NO:2, or an analog or fragment thereof, wherein the analog has amino acid sequence homology of at least 80%, alternatively of at least 90%, alternatively of at least 95%, or of 100% to SEQ ID NO:2. According to a certain embodiment, the isolated GA 2-oxidase polypeptide of kenaf has the amino acid sequence as set forth in SEQ ID NO:2.

According to yet further aspect, the present invention provides a nucleic acid molecule encoding a GA 2-oxidase of kenaf (Hibiscus cannabinus) having the nucleotide sequence as set forth in SEQ ID NO:3.

According to yet another aspect, the present invention provides a double stranded RNA molecule that down regulates expression of a GA 2-oxidase gene of Hibiscus cannabinus (kenaf) comprising:
a) a first RNA strand of the double stranded RNA comprising at least 20 contiguous nucleotides having at least 90% sequence identity to an RNA transcribed from a GA 2-oxidase gene of kenaf or a fragment thereof; and
b) a second RNA strand of said double stranded RNA comprising at least 20 contiguous nucleotides having at least 90% sequence identity to a complementary sequence of the RNA transcribed from the GA 2-oxidase gene of kenaf or a fragment thereof,
wherein the first and the second RNA strands are capable of hybridizing to each other to form said double stranded RNA molecule.

According to some embodiments, the first and second RNA strands each comprises at least 25 nucleotides, alternatively at least 50 nucleotides, alternatively at least 100 nucleotides, alternatively at least 400 nucleotides. According to further embodiments, the first RNA strand has at least 95% sequence identity, alternatively 100% sequence identity, to the RNA transcribed from the GA 2-oxidase gene of kenaf or a fragment thereof. According to yet further embodiments, the second RNA strand has at least 95% sequence identity, alternatively 100% sequence identity, to the complementary sequence of the RNA transcribed from the GA 2-oxidase gene of kenaf or a fragment thereof. According to yet further embodiments, the RNA transcribed from the GA 2-oxidase gene comprises the nucleotide sequence as set forth in SEQ ID NO:1. According to one embodiment, the first RNA strand has at least 90% sequence identity to an RNA transcribed from a nucleic acid comprising the nucleotide sequence as set forth in SEQ ID NO:4 or a fragment thereof. According to another embodiment, the second RNA strand has at least 90% sequence identity to an RNA transcribed from a nucleic acid comprising the nucleotide sequence as set forth in SEQ ID NO:38 or a fragment thereof.

According to a further aspect, the present invention provides a DNA construct for generating an RNA molecule capable of down regulating expression a GA 2-oxidase gene of kenaf, the DNA construct comprising a plant promoter operably linked to a polynucleotide sequence encoding an RNA sequence that forms a double stranded RNA, the polynucleotide sequence comprising:
(i) a first nucleotide sequence comprising at least 20 contiguous nucleotides having at least 90% sequence identity to the nucleotide sequence as set forth in SEQ ID NO:1 or a fragment thereof, and
(ii) a second nucleotide sequence comprising at least 20 contiguous nucleotides having at least 90% sequence identity to a complementary sequence of the nucleotide sequence as set forth in SEQ ID NO:1 or a fragment thereof,
wherein the RNA molecule transcribed from the DNA construct down regulates expression of GA 2-oxidase gene of kenaf.

According to some further embodiments, the first and second nucleotide sequences each comprises at least 25 nucleotides, alternatively at least 50 nucleotides, alternatively at least 100 nucleotides. According to further embodiments, the first nucleotide sequence has at least 95% sequence identity, alternatively 100% sequence homology to the nucleotide sequence as set forth in SEQ ID NO:1 or a fragment thereof. According to other embodiments, the second nucleotide sequence has at least 95% sequence identity, alternatively 100% sequence homology to the complementary sequence of the nucleotide sequence as set forth in SEQ ID NO:1 or a fragment thereof.

According to some embodiments, the first nucleotide sequence has at least 90%, alternatively 95% sequence identity to the nucleotide sequence as set forth in SEQ ID NO:4 or a fragment thereof. According to further embodiments, the second nucleotide sequence has at least 90%, alternatively 95% sequence identity to the nucleotide sequence as set forth in SEQ ID NO:38 or a fragment thereof.

According to further embodiments, the plant promoter is selected from the group consisting of constitutive promoters, inducible promoters, tissue specific promoters, and developmental stage specific promoters.

According to yet further embodiments, the DNA construct further comprises a selectable marker. According to a certain embodiment, the selectable marker is a polynucleotide sequence encoding a product conferring antibiotic resistance.

According to still further embodiments, the DNA construct further comprises an expression control sequence selected from the group consisting of an enhancer, a transcription factor, and a transcriptional terminator signal.

According to still further embodiments, the first and second nucleotide sequences of the DNA construct are operably linked to the same promoter. According to additional embodiments, the first and second nucleotide sequences are separated by a spacer sequence. According to certain embodiments, the spacer sequence is derived from an intron.

According to another aspect, the present invention provides an oligonucleotide sequence targeted to a region of the nucleotide sequence as set forth in SEQ ID NO:3 or a complementary strand thereof, wherein the oligonucleotide sequence capable of specifically hybridizing with the region of the nucleotide sequence or complementary strand thereof, thereby inhibiting expression of a GA 2-oxidase.

According to some embodiments, the region of the nucleotide sequence is selected from the group consisting of a 5′-untranslated region, coding region, stop codon region, and a 3′-untranslated region. According to certain embodiments, the oligonucleotide sequence comprises at least 20 nucleotides.

According to further embodiments, the oligonucleotide sequence comprises at least one modified internucleoside linkage. Alternatively, the oligonucleotide sequence comprises at least one modified sugar. Preferably, the oligonucleotide sequence is linked to a plant promoter, wherein the plant promoter is operably linked to the antisense oligonucleotide sequence.

According to another aspect, the present invention provides an antisense oligonucleotide sequence targeted to a region of the nucleotide sequence as set forth in SEQ ID NO:3 or a complementary strand thereof, wherein the antisense oligonucleotide sequence is capable of specifically hybridizing with the region of the nucleotide sequence or complementary strand thereof, thereby inhibiting expression of a GA 2-oxidase.

According to some embodiments, the region of the nucleotide sequence is selected from the group consisting of a 5′-untranslated region, coding region, stop codon region, and a 3′-untranslated region. According to certain embodiments, the antisense oligonucleotide sequence comprises at least 20 nucleotides. It is to be appreciated that the oligonucleotide sequence can consist of up to 500 nucleotides in length.

According to further embodiments, the antisense oligonucleotide sequence comprises at least one modified internucleoside linkage. Alternatively, the oligonucleotide sequence comprises at least one modified sugar. Preferably, the oligonucleotide sequence is linked to a plant promoter, wherein the plant promoter is operably linked to the antisense oligonucleotide sequence.

According to a further aspect, the present invention provides a nucleotide sequence encoding a ribozyme capable of cleaving specifically an RNA sequence transcribed from the nucleic acid molecule comprising the nucleotide sequence as set forth in SEQ ID NO:1.

According to another aspect, the present invention provides a plant cell transformed with a double stranded RNA of the present invention; or with a DNA construct of the present invention; or with an antisense oligonucleotide sequence of the present invention; or with a nucleotide sequence encoding a ribozyme of the present invention, wherein expression of GA 2-oxidase gene is down regulated. According to one embodiment, the plant cell is a kenaf plant cell.

According to a further aspect, the present invention provides a transgenic plant comprising at least one cell transformed with a double stranded RNA of the present invention; or with a DNA construct for generating RNAs of the present invention; or with an antisense oligonucleotide sequence of the present invention; or with a nucleotide sequence encoding a ribozyme of the present invention, wherein expression of GA 2-oxidase gene is down regulated. According to one embodiment, the transgenic plant is a kenaf plant.

According to yet further aspect, the present invention provides a seed of a transgenic plant according to the principles of the present invention. According to one embodiment, the seed of a transgenic plant is a seed from a transgenic kenaf plant.

According to another aspect, the present invention provides a stem of a transgenic plant according to the principles of the present invention. According to one embodiment, the stem of a transgenic plant is a stem from a transgenic kenaf plant.

According to yet further aspect, the present invention provides a method for producing a transgenic plant having reduced expression of GA 2-oxidase gene by down regulating GA 2-oxidase, the method comprises introducing into at least one plant cell a DNA construct comprising a plant promoter operably linked to a polynucleotide sequence encoding an RNA sequence, the polynucleotide sequence comprising:
(i) a first nucleotide sequence comprising at least 20 contiguous nucleotides having at least 90% sequence identity to a nucleic acid molecule encoding a GA 2-oxidase of the plant or a fragment thereof; and
(ii) a second nucleotide sequence comprising at least 20 contiguous nucleotides having at least 90% sequence identity to a complementary sequence of the nucleic acid molecule encoding the GA 2-oxidase of the plant or a fragment thereof,

wherein the RNA molecule transcribed from the DNA construct down regulates expression of said GA 2-oxidase gene.

According to some embodiments, the transgenic plant is selected from the group consisting of angiospermae families and gymnospermae families. According to further embodiments, the transgenic plant is selected from the group consisting of Hibiscus sp., Populus sp., Eucalyptus sp., Helianthus sp., Corchorus sp., and Boehemiera sp. According to yet further embodiments, the transgenic plant is selected from the group consisting of kenaf, Helianthus annuus L. (Sunflower), Eucaliptus camaldulensis, Cannabis sativa L., Corchorus olitorius L., and Boehemeria nivea L. According to a certain embodiment the transgenic plant is a kenaf plant. According to additional embodiments, if the transgenic plant is a kenaf plant, the DNA construct is according to the principles of the present invention.

According to other embodiments, if the transgenic plant is Arabidopsis, the first nucleotide sequence has at least 90% sequence identity to the nucleotide sequence as set forth in SEQ ID NO:35 or a fragment thereof, and the second nucleotide sequence has at least 90% sequence identity to the nucleotide sequence as set forth in SEQ ID NO:39 or a fragment thereof. According to further embodiments, if the transgenic plant is tobacco, the first nucleotide sequence has at least 90% sequence identity to the nucleotide sequence as set forth in SEQ ID NO:36 or a fragment thereof, and the second nucleotide sequence has at least 90% sequence identity to the nucleotide sequence as set forth in SEQ ID NO:40 or a fragment thereof. According to yet further embodiments, if the plant is tomato, the first nucleotide sequence has at least 90% sequence identity to the nucleotide sequence as set forth in SEQ ID NO:37 or a fragment thereof, and the second nucleotide sequence has at least 90% sequence identity to the nucleotide sequence as set forth in SEQ ID NO:41 or a fragment thereof.

According to yet further aspect, the present invention provides a method for producing a transgenic plant having reduced expression of GA 2-oxidase gene by down regulating GA 2-oxidase, the method comprises introducing into at least one plant cell a double stranded RNA of the present invention; or an antisense oligonucleotide sequence of the present invention; or a nucleotide sequence encoding a ribozyme of the present invention, thereby producing a transgenic plant having higher levels of gibberellin and lower levels of GA 2-oxidase gene expression than a non-transgenic plant. According to some embodiments, the method for producing a transgenic plant further comprises a step of introducing into the transgenic plant a fiber-increasing effective amount of a gibberellin or/and auxin. According to additional embodiments, the method for producing a transgenic plant further comprises a step of introducing into at least one plant cell a nucleic acid molecule encoding gibberellin 20-oxidase. According to one embodiment the transgenic plant is kenaf.

According to some embodiments, introducing the double stranded RNAs of the present invention; or the DNA constructs of the present invention; or the antisense oligonucleotide sequences of the present invention; or the nucleotide sequences encoding a ribozyme of the present invention, is performed by a method selected from the group consisting of Agrobacterium-mediated transformation, microprojectile bombardment, pollen mediated transformation, plant RNA virus mediated transformation, liposome mediated transformation, direct gene transfer, and electroporation of compact embryogenic calli.

According to further aspect, the present invention provides a method for increasing the fiber crop of a plant comprising introducing into at least one plant cell a DNA construct comprising a plant promoter operably linked to a polynucleotide sequence encoding an RNA sequence, the polynucleotide sequence comprising:
(i) a first nucleotide sequence comprising at least 20 contiguous nucleotides having at least 90% sequence identity to a nucleic acid molecule encoding a GA 2-oxidase of the plant or a fragment thereof; and
(ii) a second nucleotide sequence comprising at least 20 contiguous nucleotides having at least 90% sequence identity to a complementary sequence of the nucleic acid molecule encoding the GA 2-oxidase of said plant or a fragment thereof,
wherein the RNA transcribed from the DNA construct down regulates expression of GA 2-oxidase gene.

According to some embodiments, the first nucleotide sequence has at least 95%, alternatively 100%, sequence identity to the nucleic acid molecule encoding said GA 2-oxidase or a fragment thereof. According to additional embodiments, the second nucleotide sequence has at least 95%, alternatively 100%, sequence identity or 100% homology to the complementary sequence of the nucleic acid molecule encoding said GA 2-oxidase or a fragment thereof.

According to further embodiments, the plant is selected from the group consisting of angiospermae families and gymnospermae families. According to additional embodiments, the angiospermae families include, but are not limited to, Hibiscus sp., Populus sp., Eucalyptus sp., Helianthus sp., Corchorus sp., and Boehemeria sp. According to additional embodiments, the plant is selected from the group consisting of kenaf, Helianthus annuus L. (Sunflower), Eucaliptus camaldulensis, Cannabis sativa L., Corchorus olitorius L., and Boehemeria nivea L. According to a certain embodiment, the plant is a kenaf plant. According to additional embodiments, if the transgenic plant is a kenaf plant, the DNA construct is according to the principles of the present invention.

According to other embodiments, if the transgenic plant is Arabidopsis, the first nucleotide sequence has at least 90% sequence identity to the nucleotide sequence as set forth in SEQ ID NO:35 or a fragment thereof, and the second nucleotide sequence has at least 90% sequence identity to the nucleotide sequence as set forth in SEQ ID NO:39 or a fragment thereof. According to further embodiments, if the transgenic plant is tobacco, the first nucleotide sequence has at least 90% sequence identity to the nucleotide sequence as set forth in SEQ ID NO:36 or a fragment thereof, and the second nucleotide sequence has at least 90% sequence identity to the nucleotide sequence as set forth in SEQ ID NO:40 or a fragment thereof. According to yet further embodiments, if the plant is tomato, the first nucleotide sequence has at least 90% sequence identity to the nucleotide sequence as set forth in SEQ ID NO:37 or a fragment thereof, the second nucleotide sequence has at least 90% sequence identity to the nucleotide sequence as set forth in SEQ ID NO:41 or a fragment thereof.

According to another aspect, the present invention provides an expression vector comprising an isolated nucleic acid molecule comprising the nucleotide sequence as set forth in SEQ ID NO:1 encoding a GA 2-oxidase of kenaf, or a complementary strand or a homologous sequence thereto, wherein the homologous sequence has sequence identity of at least 80% to SEQ ID NO:1. According to some embodiments, the homologous sequence within the expression vector has sequence identity of at least 90%, alternatively of at least 95%, alternatively of 100% to SEQ ID NO: 1. According to a certain embodiment, the expression vector comprises an isolated nucleic acid molecule having the nucleotide sequence as set forth in SEQ ID NO:1 which encodes a GA 2-oxidase of kenaf.

According to a further aspect, the present invention provides a plant cell having over expression of a GA 2-oxidase gene of Kenaf, the plant cell being transformed with an expression vector comprising an isolated nucleic acid molecule comprising the nucleotide sequence as set forth in SEQ ID NO:1 which encodes a GA 2-oxidase of kenaf, or a complementary strand or a homologous sequence thereto, wherein the homologous sequence has sequence identity of at least 80% to SEQ ID NO:1. According to some embodiments, the homologous sequence within the expression vector has sequence identity of at least 90%, alternatively of at least 95%, alternatively of 100% to SEQ ID NO:1. According to a certain embodiment, the plant cell is transformed with an expression vector comprising an isolated nucleic acid molecule comprising the nucleotide sequence as set forth in SEQ ID NO:1, which encodes a GA 2-oxidase of kenaf. According to one embodiment, the plant cell is a Kenaf plant cell.

According to yet further aspect, the present invention provides a transgenic plant comprising at least one plant cell transformed with an expression vector comprising an isolated nucleic acid molecule comprising the nucleotide sequence as set forth in SEQ ID NO:1 which encodes a GA 2-oxidase of kenaf, or a complementary strand or a homologous sequence thereto, wherein the homologous sequence has sequence identity of at least 80% to SEQ ID NO:1. According to some embodiments, the homologous sequence has sequence identity of at least 90%, alternatively of at least 95%, alternatively of 100% to SEQ ID NO:1. According to a certain embodiment, the transgenic plant comprises at least one plant cell transformed with an expression vector comprising an isolated nucleic acid molecule comprising the nucleotide sequence as set forth in SEQ ID NO:1 which encodes a GA 2-oxidase of kenaf. According to one embodiment, the transgenic plant is a Kenaf plant.

According to another aspect, the present invention provides a method for producing a transgenic plant having over-expression of a GA 2-oxidase of Kenaf, the method comprising introducing into at least one cell of a plant an expression vector comprising an isolated nucleic acid molecule comprising the nucleotide sequence as set forth in SEQ ID NO:1 which encodes a GA 2-oxidase of kenaf, or a complementary strand or a homologous sequence thereto, wherein the homologous sequence has sequence identity of at least 80%, alternatively of at least 90%, alternatively of at least 95%, alternatively of 100% to SEQ ID NO:1, thereby producing a transgenic plant having lower levels of gibberellin (GA) than a non-transgenic plant. According to a certain embodiment, the method for producing a transgenic plant comprises the step of introducing into at least one cell an expression vector comprising an isolated nucleic acid molecule comprising the nucleotide sequence as set forth in SEQ ID NO:1 which encodes a GA 2-oxidase of kenaf. According to one embodiment, the transgenic plant is a Kenaf plant.

These and other embodiments of the present invention will be better understood in relation to the figures, description, examples and claims that follow.

### BRIEF DESCRIPTION OF THE FIGURES

FIGS. 1A-B show photographs of Arabidopsis plants grown under long day or short day conditions. FIG. 1A shows Arabidopsis plants grown under long day conditions; Left: wild type (wt) control; middle: GA 20-Oxidase transgenic over-expressed plant; right: GA 2-Oxidase transgenic silenced plant. FIG. 1B shows Arabidopsis plants grown under short day conditions. Left: wt control; right: GA 20-Oxidase transgenic over-expressed Arabidopsis.

FIG. 2 shows stem cross sections of wild type and transgenic Arabidopsis plants. In the transgenic GA 20-oxidase over expressed plants an increase in fiber cell number in vascular bundles and between the bundles was observed as well as reduced lignification of the fiber cell walls.

FIG. 3 shows a photograph of tobacco plants. A wild type tobacco plant and a GA 20-oxidase transgenic over-expressed plant are shown.

FIGS. 4A-C show stem cross sections of wild type and transgenic GA 20-oxidase plants. Inner phloem islands of GA-20 Oxidase over expressing plants contain fibers (FIGS. 4A and 4B) which were not observed in the internodes of wt plants (FIG. 4C).

FIGS. 5A-C show photographs of wild type and GA 2-oxidase silenced tobacco plants. Left: transgenic silenced GA 2-oxidase plants; right: wild type plants. The transgenic silenced GA 2-oxidase plants are longer than wild type plants.

FIGS. 6A-B show comparison between GA 2-Oxidase silenced lines vs. wt control. FIG. 6A shows comparison of the mean number of internodes, mean length of longest leaf and average heights of the GA 2-Oxidase silenced lines vs. wt control. FIG. 6B shows mean, shortest and longest heights (cm) for each GA 2-Oxidase silenced plant and wt plant.

FIG. 7 shows the genomic sequence of a GA 2-oxidase of kenaf.

FIG. 8 shows the nucleotide sequence of the cDNA encoding GA 2-oxidase of kenaf and the amino acid sequence of the enzyme.

### DETAILED DESCRIPTION OF PREFERRED EMBODIMENTS

### Definitions

The term “gene” refers to chromosomal DNA, plasmid DNA, cDNA, synthetic DNA, or other DNA that encodes a peptide, polypeptide, protein, or RNA molecule, and regions flanking the coding sequence involved in the regulation of expression.

The term “nucleic acid” refers to deoxyribonucleic acid (DNA) and ribonucleic acid (RNA). The nucleic acid can be a single, double, or multiple stranded and can comprise modified or unmodified nucleotides.

The term “plant” is used herein in its broadest sense. It includes, but is not limited to, any species of woody, herbaceous, perennial or annual plant. It also refers to a plurality of plant cells that are largely differentiated into a structure that is present at any stage of a plant development. Such structures include, but are not limited to, a root, stem, shoot, leaf, flower, petal, fruit, etc.

The term “hybridization” refers to the ability of a strand of nucleic acid to join with a complementary strand via base pairing. Hybridization occurs when complementary sequences in the two nucleic acid strands bind to one another.

As used herein, the term “homology” when used in relation to nucleic acid sequences refers to a degree of similarity or identity between at least two nucleotide sequences. There may be partial homology or complete homology (i.e., identity). “Sequence identity” refers to a measure of relatedness between two or more nucleotide sequences, expressed as a percentage with reference to the total comparison length. The identity calculation takes into account those nucleotide residues that are identical and in the same relative positions in their respective sequences. A gap, i.e. a position in an alignment where a residue is present in one sequence but not in the other is regards as a position with non-identical residues. A widely used and accepted computer program for performing sequence alignments is CLUSTALW v1.6 (Thompson, et al. Nucl. Acids Res., 22: 4673-4680, 1994). Similarly, term “homology” when used in relation to protein sequences refers to a degree of similarity or identity between at least two protein sequences. There may be partial homology or complete homology (i.e., identity).

The term “promoter” or “promoter region” refers to a nucleic acid sequence, usually found upstream (5′) to a coding sequence that controls expression of the coding sequence by controlling production of messenger RNA (mRNA) by providing the recognition site for RNA polymerase and/or other factors necessary for start of transcription at the correct site. As contemplated herein, a promoter or promoter region includes variations of promoters derived by means of ligation to various regulatory sequences, random or controlled mutagenesis, and addition or duplication of enhancer sequences. The promoter region disclosed herein, and biologically functional equivalents thereof, are responsible for driving the transcription of coding sequences under their control when introduced into a host as part of a suitable recombinant vector, as demonstrated by its ability to produce mRNA.

The term “DNA construct” or “expression vector” refers to any agent such as a plasmid, cosmid, virus, autonomously replicating sequence, phage, or linear or circular single-stranded or double-stranded DNA or RNA nucleotide sequence, derived from any source, capable of genomic integration or autonomous replication, comprising a DNA molecule in which one or more DNA sequences have been linked in a functionally operative manner. Such DNA constructs or vectors are capable of introducing a 5′ regulatory sequence or promoter region and a DNA sequence for a selected gene product into a cell in such a manner that the DNA sequence is transcribed into a functional mRNA which is translated and therefore expressed. DNA constructs or expression vectors may be constructed to be capable of expressing double stranded RNAs or antisense RNAs in order to inhibit translation of a specific RNA of interest.

The term “heterologous gene” refers to a gene encoding a polypeptide or protein that is not in its natural environment (i.e., has been altered by the hand of man). For example, a heterologous gene includes a gene from one species introduced into another species. A heterologous gene also includes a gene native to an organism that has been altered in some way (e.g., mutated, added in multiple copies, linked to a non-native promoter or enhancer sequence, etc.). Heterologous genes may comprise plant gene sequences that comprise cDNA forms of a plant gene; the cDNA sequences may be expressed in either a sense (to produce mRNA) or anti-sense orientation (to produce an anti-sense RNA transcript that is complementary to the mRNA transcript).

The term “transgenic” when used in reference to a plant or fruit or seed (i.e., a “transgenic plant” or “transgenic fruit” or a “transgenic seed”) refers to a plant or fruit or seed that contains at least one heterologous gene in one or more of its cells.

The term “antisense oligonucleotide” refers to an oligonucleotide that binds to target RNA by means of RNA-RNA or RNA-DNA or RNA-PNA (protein nucleic acid) interactions and alters the activity of the target RNA (for a review, see Stein and Cheng, 1993 Science 261, 1004). Typically, antisense molecules are complementary to a target sequence along a single contiguous sequence of the antisense molecule. However, in certain embodiments, antisense oligonucleotide can be complementary to two (or even more) non-contiguous target sequences.

The term “transformation” refers to a process of introducing an exogenous nucleic acid sequence (e.g., a vector, expression vector) into a cell or protoplast in which that exogenous nucleic acid is incorporated into a chromosome or is capable of autonomous replication.

The term “regeneration” refers to the process of growing a plant from a plant cell (e.g., plant protoplast or explant).

The term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and into protein, through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease the production of gene expression products (i.e., RNA or protein). Molecules (e.g., transcription factors) that are involved in up-regulation or down-regulation are often called “activators” and “repressors,” respectively.

The term “fragment” refers to a polynucleotide or polypeptide which is a portion of a full length nucleic acid molecule or a full length protein, respectively.

The term “analog” as used herein refers to a protein comprising altered sequences of GA 2-oxidase of kenaf of SEQ ID NO:2 by amino acid substitutions, additions, or chemical modifications. By using “amino acid substitutions”, it is meant that functionally equivalent amino acid residues are substituted for residues within the sequence resulting in a silent change. For example, one or more amino acid residues within the sequence can be substituted by another amino acid of a similar polarity, which acts as a functional equivalent, resulting in a silent alteration. Such substitutions are known as conservative substitutions. Additionally, a non-conservative substitution may be made in an amino acid so long as the enzymatic activity of the analog is preserved if compared to the activity of an intact GA 2-oxidase of kenaf.

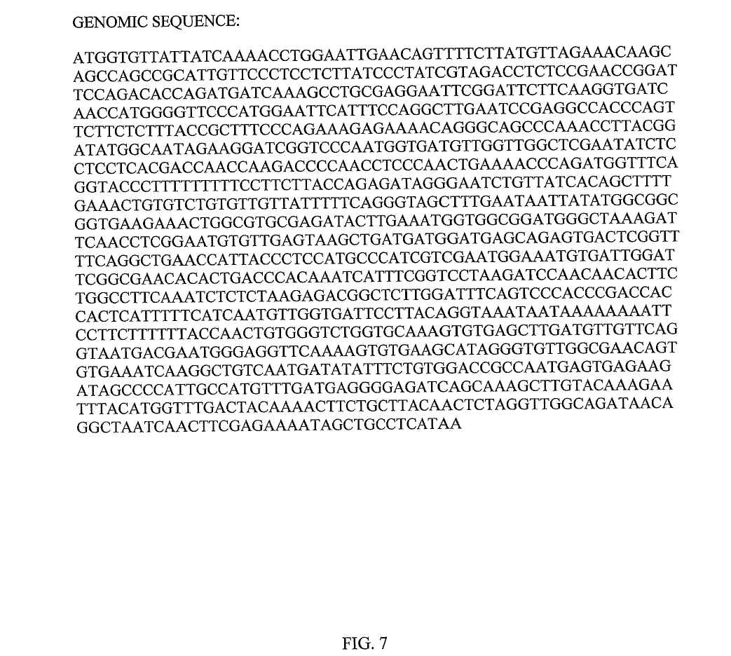

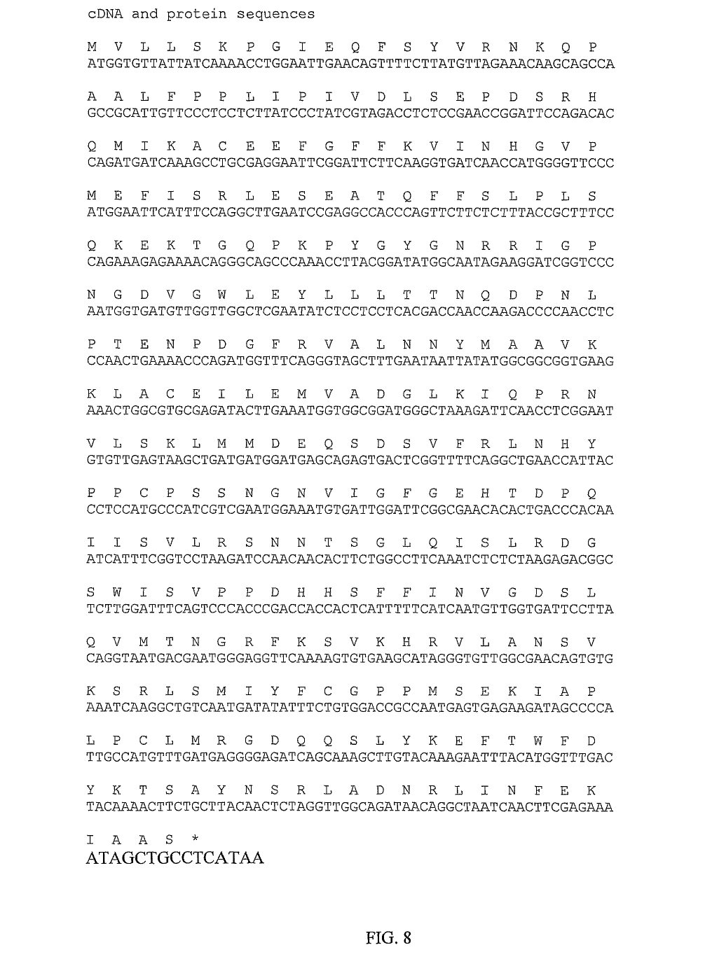

The present invention provides the cDNA and amino acid sequence of a GA 2-oxidase of kenaf as set forth in SEQ ID NO:1 and SEQ ID NO:2 (FIG. 8). The present invention further provides the genomic sequence of a GA 2-oxidase of kenaf as set forth in SEQ ID NO:3 (FIG. 7) and a DNA sequence for silencing GA 2-oxidase expression as set forth in SEQ ID NO:4.

The present invention provides a DNA construct or an expression vector comprising a nucleic acid of interest which may further comprise a plant promoter.

A number of promoters which are active in plant cells have been described in the literature. These include the nopaline synthase (NOS) promoter (Ebert et al., Proc. Natl. Acad. Sci. U.S.A. 84: 5745-5749, 1987), the octopine synthase (OCS) promoter, the caulimovirus promoters such as the cauliflower mosaic virus (CaMV) 19S promoter (Lawton et al., Plant Mol. Biol. 9: 315-324, 1987) and the CaMV ³⁵S promoter (Odell et al., Nature 313: 810-812, 1985), the figwort mosaic virus 35S-promoter; the light-inducible promoter from the small subunit of ribulose-1,5-bis-phosphate carboxylase (ssRUBISCO), the Adh promoter (Walker et al., Proc. Natl. Acad. Sci. U.S.A. 84: 6624-6628, 1987), the sucrose synthase promoter (Yang et al., Proc. Natl. Acad. Sci. USA. 87: 4144-4148, 1990), the R gene complex promoter (Chandler et al., The Plant Cell 1: 1175-1183, 1989), and the chlorophyll binding protein gene promoter, and the like. These promoters have been used to create DNA constructs which have been expressed in plants.

For the purpose of expression in source tissues of the plant, such as the leaf, seed, root or stem, it is preferred that the promoters utilized in the present invention have relatively high expression in these specific tissues. For this purpose, one may choose from a number of promoters for genes with tissue- or cell-specific or -enhanced expression. Examples of such promoters reported in the literature include the chloroplast glutamine synthetase GS2 promoter from pea (Edwards et al., Proc. Natl. Acad. Sci. U.S.A. 87: 3459-3463, 1990), the chloroplast fructose-1,6-biphosphatase (FBPase) promoter from wheat (Lloyd et al., Mol. Gen. Genet. 225: 209-216, 1991), the nuclear photosynthetic ST-LS1 promoter from potato (Stockhaus et al., EMBO J. 8: 2445-2451, 1989), the serine/threonine kinase (PAL) promoter and the glucoamylase (CHS) promoter from Arabidopsis thaliana.

Expression of specific promoters can be monitored by use of a reporter gene such as β-glucuronidase (Jefferson et, EMBO J. 6: 3901-3907, 1987), luciferase (LUC, Ow et al., Science 234: 856-859, 1986), green fluorescent protein (GFP, Sheen et al., Plant J. 8: 777-784, 1995) or any other reporter gene cloned downstream of the promoter and transiently or stably transformed into plant cells. Detection of reporter gene activity is indicative of transcriptional activity of the promoter within the tissue.

The DNA constructs or vectors may also include with the coding region of interest a nucleic acid sequence that acts, in whole or in part, to terminate transcription of that region. For example, such sequences have been isolated including the Tr7 3′ sequence and the NOS 3′ sequence (Ingelbrecht et al., The Plant Cell 1: 671-680, 1989; Bevan et al., Nucleic Acids Res. 11: 369-385, 1983), or the like.

The DNA constructs or vectors may also include regulatory elements. Examples of such include the Adh intron 1 (Callis et al., Genes and Develop. 1: 1183-1200, 1987), the sucrose synthase intron (Vasil et al., Plant Physiol. 91: 1575-1579, 1989), first intron of the maize hsp70 gene (U.S. Pat. No. 5,362,865), and the TMV omega element (Gallie et al., The Plant Cell 1: 301-311, 1989). These and other regulatory elements may be included when appropriate.

The DNA constructs or vectors may also include a selectable marker. Selectable markers may be used to select for plants or plant cells that contain the exogenous genetic material. Examples of such include a neo gene (Potrykus et al., Mol. Gen. Genet. 199: 183-188, 1985) which codes for kanamycin resistance and can be selected for using kanamycin, G418; a bar gene which codes for bialaphos resistance; a mutant EPSP synthase gene which encodes glyphosate resistance; a nitrilase gene which confers resistance to bromoxynil (Stalker et al., J. Biol. Chem. 263: 6310-6314, 1988); and a methotrexate resistant DHFR gene (Thillet et al., J. Biol. Chem. 263: 12500-12508, 1988).

The DNA construct or expression vector may also include translational enhancers. DNA constructs could contain one or more 5′ non-translated leader sequences which may serve to enhance expression of the gene products from the resulting mRNA transcripts. Such sequences may be obtained from viral RNAs, from suitable eukaryotic genes, or from a synthetic gene sequence, and the like.

### Plant Transformation

The DNA construct of the present invention can be utilized to stably or transiently transform plant cells. In stable transformation, the nucleic acid molecule is integrated into the plant genome, and as such it represents a stable and inherited trait. In transient transformation, the nucleic acid molecule is expressed by the cell transformed but is not integrated into the genome, and as such represents a transient trait.

The principal methods of the stable integration of exogenous DNA into plant genomic DNA include: (i) Agrobacterium-mediated gene transfer (see for example Klee et al. (1987). Annu Rev Plant Physiol 38, 467-486; and Gatenby, A. A. Plant Biotechnology, pp. 93-112 (1989) S. Kung and C. J. Arntzen, eds., Butterworth Publishers, Boston, Mass.); and (ii) Direct DNA transfer include microinjection, electroporation, and microprojectile bombardment (U.S. Pat. No. 6,723,897 and references therein, which is incorporated by reference as if fully set forth herein).

Agrobacterium-mediated transfer is a widely applicable system for introducing genes into plant cells because the DNA can be introduced into whole plant tissues, thereby bypassing the need for regeneration of an intact plant from a protoplast. The Agrobacterium-mediated system includes the use of plasmid vectors that contain defined DNA segments which integrate into the plant genomic DNA. Methods of inoculation of the plant tissue vary depending upon the plant species and the Agrobacterium delivery system. A widely used approach is the leaf-disc procedure, which can be performed with any tissue explant that provides a good source for initiation of whole-plant differentiation. A supplementary approach employs the Agrobacterium delivery system in combination with vacuum infiltration. The Agrobacterium system is especially useful for in the creation of transgenic dicotyledenous plants.

In electroporation, the protoplasts are briefly exposed to a strong electric field, opening up mini-pores to allow DNA to enter. In microinjection, the DNA is mechanically injected directly into the cells using micropipettes. In microparticle bombardment, the DNA is adsorbed on microprojectiles such as magnesium sulfate crystals or tungsten particles, and the microprojectiles are physically accelerated into cells or plant tissues.

Following stable transformation, plant propagation then occurs. The most common method of plant propagation is by seed. The disadvantage of regeneration by seed propagation, however, is the lack of uniformity in the crop due to heterozygosity, since seeds are produced by plants according to the genetic variances governed by Mendelian rules. In other words, each seed is genetically different and each will grow with its own specific traits. Therefore, it is preferred that the regeneration be effected such that the regenerated plant has identical traits and characteristics to those of the parent transgenic plant. The preferred method of regenerating a transformed plant is by micropropagation, which provides a rapid, consistent reproduction of the transformed plants.

Micropropagation is a process of growing second-generation plants from a single tissue sample excised from a selected parent plant or cultivar. This process permits the mass reproduction of plants having the preferred tissue and expressing a fusion protein. The newly generated plants are genetically identical to, and have all of the characteristics of, the original plant. Micropropagation allows for mass production of quality plant material in a short period of time and offers a rapid multiplication of selected cultivars with preservation of the characteristics of the original transgenic or transformed plant. The advantages of this method of plant cloning include the speed of plant multiplication and the quality and uniformity of the plants produced.

Micropropagation is a multi-stage procedure that requires alteration of culture medium or growth conditions between stages. The micropropagation process involves four basic stages: stage one, initial tissue culturing; stage two, tissue culture multiplication; stage three, differentiation and plant formation; and stage four, greenhouse culturing and hardening. During stage one, the tissue culture is established and certified contaminant-free. During stage two, the initial tissue culture is multiplied until a sufficient number of tissue samples are produced to meet production goals. During stage three, the newly grown tissue samples are divided and grown into individual plantlets. At stage four, the transformed plantlets are transferred to a greenhouse for hardening where the plants' tolerance to light is gradually increased so that they can continue to grow in the natural environment.

Although stable transformation is presently preferred, transient transformation of, for instance, leaf cells, meristematic cells, or the whole plant is also envisaged by the present invention.

Transient transformation can be effected by any of the direct DNA transfer methods described above or by viral infection using modified plant viruses.

Viruses that have been shown to be useful for the transformation of plant hosts include cauliflower mosaic virus (CaMV), tobacco mosaic virus (TMV), and baculovirus (BV). Transformation of plants using plant viruses is described in, for example, Communications in Molecular Biology: Viral Vectors, Cold Spring Harbor Laboratory, New York, pp. 172-189.

Construction of plant RNA viruses for the introduction and expression of non-viral exogenous nucleic acid sequences in plants is known in the art and demonstrated by the above references as well as by Dawson et al. (1989) Virology 172, 285-292.

If the transforming virus is a DNA virus, one skilled in the art may make suitable modifications to the virus itself. Alternatively, the virus can first be cloned into a bacterial plasmid for ease of constructing the desired viral vector with the foreign DNA. The virus can then be excised from the plasmid. If the virus is a DNA virus, a bacterial origin of replication can be attached to the viral DNA, which is then replicated by the bacteria. Transcription and translation of the DNA will produce the coat protein, which will encapsidate the viral DNA. If the virus is an RNA virus, the virus is generally cloned as a cDNA and inserted into a plasmid. The plasmid is then used to make all of the plant genetic constructs. The RNA virus is then transcribed from the viral sequence of the plasmid, followed by translation of the viral genes to produce the coat proteins which encapsidate the viral RNA.

Transformation of plant protoplasts has been reported using methods based on calcium phosphate precipitation, polyethylene glycol treatment, electroporation, and combinations of these treatments (see, for example, Marcotte et al., Nature 335: 454-457, 1988).

The regeneration, development, and cultivation of plants from single plant protoplast transformants or from various transformed explants is well known in the art This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

The development or regeneration of plants containing the foreign, exogenous gene that encodes a protein of interest is well known in the art. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present invention containing a desired polypeptide is cultivated using methods well known to one skilled in the art.

### Silencing the Expression of GA 2-Oxidase

Silencing of GA 2-oxidase can be effected on the genomic and/or the transcript level using a variety of molecules that interfere with transcription and/or translation (e.g., antisense, siRNA, Ribozyme, or DNAzyme).

One agent capable of down regulating or silencing a GA 2-oxidase is a small interfering RNA (siRNA) molecule in the process of RNA interference (RNAi). RNAi is a two-step process. In the first, the initiation step, input double-stranded (dsRNA) is digested into 21- to 23-nucleotide (nt) small interfering RNAs (siRNAs), probably by the action of Dicer, a member of the RNase III family of dsRNA-specific ribonucleases, which processes (cleaves) dsRNA (introduced directly or by means of a transgene or a virus) in an ATP-dependent manner. Successive cleavage events degrade the RNA to 19- to 21-bp duplexes (the siRNA), each with 2-nucleotide 3′ overhangs.

In the second step, termed the effector step, the siRNA duplexes bind to a nuclease complex to form the RNA-induced silencing complex (RISC). An ATP-dependent unwinding of the siRNA duplex is required for activation of the RISC. The active RISC then targets the homologous transcript by base-pairing interactions and cleaves the mRNA into 12-nucleotide fragments from the 3′ terminus of the siRNA. Although the mechanism of cleavage remains to be elucidated, research indicates that each RISC contains a single siRNA and an RNase.

Because of the remarkable potency of RNAi, an amplification step within the RNAi pathway has been suggested. Amplification could occur by copying of the input dsRNAs to generate more siRNAs, or by replication of the siRNAs formed. Alternatively or additionally, amplification could be effected by multiple turnover events of the RISC. For more information on RNAi, see the following reviews: Cullen, B. R. (2002). RNA interference: antiviral defense and genetic tool. Nat Immunol 3, 597-599; and Brantl, S. (2002). Antisense-RNA regulation and RNA interference. Biochim Biophys Acta 1575, 15-25.

Synthesis of RNAi molecules suitable for use with the present invention can be affected as follows. First, the GA 2-oxidase mRNA sequence is scanned downstream of the AUG start codon for AA-dinucleotide sequences. Occurrence of each AA and the 19 3′-adjacent nucleotides is recorded as a potential siRNA target site. Preferably, siRNA target sites are selected from the open reading frame (ORF), as non-translated regions (UTRs) are richer in regulatory protein binding sites. UTR-binding proteins and/or translation initiation complexes may interfere with binding of the siRNA endonuclease complex (Tuschl (2001)). It will be appreciated, however, that siRNAs directed at non-translated regions may also be effective, as demonstrated for GAPDH, wherein siRNA directed at the 5′ UTR mediated about a 90% decrease in cellular GAPDH mRNA and completely abolished protein levels.

Second, potential target sites are compared to an appropriate genomic database using any sequence alignment software, such as the BlastN software available from the NCBI server (www.ncbi.nlm.nih.gov/BLAST/). Putative target sites that exhibit significant homology to other coding sequences are filtered out.

Qualifying target sequences are selected as templates for siRNA synthesis. Preferred sequences are those including low G/C content, as these have proven to be more effective in mediating gene silencing as compared with sequences including G/C content higher than 55%. Several target sites are preferably selected along the length of the target gene for evaluation. For better evaluation of the selected siRNAs, a negative control is preferably used in conjunction. Negative-control siRNAs preferably include the same nucleotide composition as the siRNAs but lack significant homology to the genome. Thus, a scrambled nucleotide sequence of the siRNA is preferably used, provided it does not display any significant homology to any other gene.

The present invention provides a DNA construct designed for generating siRNAs targeted to a GA 2-oxidase of kenaf. According to one embodiment, the DNA construct comprises:

(a) at least one plant promoter operably linked to;

(b) a polynucleotide sequence encoding an RNA sequence that forms a double stranded RNA, wherein the polynucleotide sequence comprises a first nucleotide sequence of at least 20 contiguous nucleotides having at least 90% sequence identity to the sense nucleotide sequence of the GA 2-oxidase or a fragment thereof, and a second nucleotide sequence of at least 20 contiguous nucleotides having at least 90% sequence identity to the complementary sequence of the sense nucleotide sequence of said GA 2-oxidase or a fragment thereof; and optionally

(c) a transcription termination signal.

According to some embodiments, the DNA construct according to the present invention is designed to express a stem-loop RNA, comprising further to the first (sense) and the second (antisense) nucleotide sequences a spacer polynucleotide sequence, located between the DNA region encoding the first and the second nucleotide sequences. The length of the spacer polynucleotide sequence may vary according to the specific structure of the stem-loop RNA. Typically, the ratio of the spacer length to the first and second nucleotide sequences length is in the range of 1:5 to 1:10.

According to another embodiment, the DNA construct designed for generating siRNAs targeted to a GA 2-oxidase of kenaf comprises:

(a) at least one plant promoter operably linked to;

(b) a polynucleotide sequence encoding an RNA sequence that forms a double stranded RNA in the form of stem-loop, wherein the double stranded RNA comprises a first nucleotide sequence of at least 20 contiguous nucleotides having at least 90% sequence identity to the sense nucleotide sequence of the GA 2-oxidase or a fragment thereof; a second nucleotide sequence of at least 20 contiguous nucleotides having at least 90% identity to the complementary sequence of the sense nucleotide sequence of the GA 2-oxidase or a fragment thereof;

(c) a spacer sequence connecting the first and second nucleotide sequence; and optionally,

(d) a transcription termination signal.

According to one embodiment, the spacer comprises a nucleotide sequence derived from a gene intron known in the art to enhance the production of siRNAs.

The first and second nucleotide sequences of the double stranded RNA of the present invention can be a fragment of the GA 2-oxidase gene, can be a full-length gene, a non-coding region or part thereof or a combination of same.

As used herein, nucleotide sequence of RNA molecule may be identified by reference to a DNA nucleotide sequence of the sequence listing. However, the person skilled in the art will understand whether RNA or DNA is meant depending on the context. Furthermore, the nucleotide sequence is identical except that the T-base is replaced by uracil (U) in RNA molecule.

According to certain embodiments, the DNA construct designed for generating siRNAs targeted to a GA 2-oxidase or a fragment thereof, the length of the second (antisense) nucleotide sequence of the DNA construct is largely determined by the length of the first (sense) nucleotide sequence, and may correspond to the length of the latter sequence. However, it is possible to use antisense sequences that differ in length by about 10%.

The first and the second nucleotide sequences of the DNA construct designed for generating siRNAs can be of any length providing the sequences comprising at least 20 contiguous nucleotides. Thus, the first and the second nucleotide sequences can comprise a fragment of a GA 2-oxidase gene or a full length of the GA 2-oxidase gene. According to some embodiments, the length of the nucleotide sequences is from 20 nucleotides to 1,200 nucleotides.

According to one embodiment, the siRNAs generated by the DNA construct of the present invention are targeted to silence a fragment of a GA 2-oxidase gene, including the coding region for GA 2-oxidase.

According to one preferred embodiment, the first nucleotide sequence comprises a nucleotide sequence having 90% identity, suitably 95% or 100% identity to the nucleotide sequence set forth in SEQ ID NO:1 or a fragment thereof. According to a certain embodiment, the first nucleotide sequence consists of the sequence as set forth in SEQ ID NO:4.

According to another preferred embodiment, the second nucleotide sequence comprises a nucleotide sequence having 90% identity, suitably 95% or 100% identity to the complementary strand of the nucleic acid molecule set forth in SEQ ID NO:1 or a fragment thereof. According to a certain embodiment, the second nucleotide sequence is complementary to the sequence set forth in SEQ ID NO:4.

According to certain embodiments, the first and the second nucleotide sequences are operably linked to the same promoter. The transcribed strands, which are at least partially complementary, are capable of forming dsRNA. According to other embodiments, the first and the second nucleotide sequences are transcribed as two separate strands. When the dsRNA is thus produced, the DNA sequence to be transcribed is flanked by two promoters, one controlling the transcription of the first nucleotide sequence, and the other controlling the transcription of the second, complementary nucleotide sequence. These two promoters may be identical or different. According to one embodiment, the first and the second nucleotide sequences are operably linked to the same promoter.

Another agent capable of silencing a GA 2-oxidase is a DNAzyme molecule, which is capable of specifically cleaving an mRNA transcript or a DNA sequence of the GA 2-oxidase. DNAzymes are single-stranded polynucleotides that are capable of cleaving both single- and double-stranded target sequences (Breaker et al. (1995) Curr Biol 2, 655-660; Santoroet al. (1997) Proc Natl Acad Sci USA 94, 4262-4266). A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (for review of DNAzymes, see: Khachigian (2002) Curr Opin Mol Ther 4, 119-121).

Examples of construction and amplification of synthetic, engineered DNAzymes recognizing single- and double-stranded target cleavage sites are disclosed in U.S. Pat. No. 6,326,174 to Joyce et al.

Silencing of a GA 2-oxidase can also be affected by using an antisense oligonucleotide capable of specifically hybridizing with an mRNA transcript encoding the GA 2-oxidase.

It is to be understood that algorithms are available for identifying those sequences with the highest predicted binding affinity for their target mRNA based on a thermodynamic cycle that accounts for the energetics of structural alterations in both the target mRNA and the oligonucleotide (see, for example, Walton et al. (1999) Biotechnol Bioeng 65, 1-9).

In addition, several approaches for designing and predicting efficiencies of specific oligonucleotides using an in vitro system were also published (Matveeva et al. (1998). Nature Biotechnology 16, 1374-1375).

Thus, the current consensus is that recent developments in the field of antisense technology, which, as described above, have led to the generation of highly accurate antisense design algorithms and a wide variety of oligonucleotide delivery systems, enable an ordinarily skilled artisan to design and implement antisense approaches suitable for down regulating expression of gene sequences without having to resort to undue trial and error experimentation.

Another agent capable of down regulating a GA 2-oxidase is a ribozyme molecule capable of specifically cleaving an mRNA transcript encoding a GA 2-oxidase. Ribozymes increasingly are being used for the sequence-specific inhibition of gene expression by the cleavage of mRNAs encoding proteins of interest (Welch et al. (1998) Curr Opin Biotechnol 9, 486-496). The possibility of designing ribozymes to cleave any specific target RNA has rendered them valuable tools in both basic research and therapeutic applications.

An additional method of regulating the expression of a GA 2-oxidase gene in cells is via triplex-forming oligonucleotides (TFOs). Recent studies show that TFOs can be designed to recognize and bind to polypurine or polypirimidine regions in double-stranded helical DNA in a sequence-specific manner. These recognition rules are outlined in: Maher III et al. (1989) Science 245, 725-730; Cooney et al. (1988) Science 241, 456-459). Modifications of the oligonucleotides, such as the introduction of intercalators and backbone substitutions, and optimization of binding conditions (e.g., pH and cation concentration) have aided in overcoming inherent obstacles to TFO activity such as charge repulsion and instability, and it was recently shown that synthetic oligonucleotides can be targeted to specific sequences (for a recent review, see Seidman et al. (2003) J Clin Invest 112, 487-494).

Thus, a triplex-forming sequence may be devised for any given sequence in the GA 2-oxidase regulatory region. Triplex-forming oligonucleotides preferably are at least 19, more preferably 25, still more preferably 30 or more, nucleotides in length, up to 50 base pairs.

It is to be understood that any of the silencing nucleotide sequences of the present invention can comprise at least one 2′-sugar modification, at least one nucleic acid base modification, and/or at least one phosphate backbone modification.

Down regulation of gene expression refers to the decrease or absence in the level of protein and/or mRNA product of a target gene, i.e., GA 2-oxidase gene. The consequences of down regulation can be confirmed by examination of the outward properties of the cell or plant or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), and fluorescence activated cell analysis (FACS). For RNA-mediated inhibition in cells, gene expression is conveniently assayed by use of a reporter or drug resistance gene whose protein product is easily assayed.

The transgenic plants comprising a silencing nucleotide sequence as disclosed in the present invention can be used in various industries in the production of paper, textiles, ropes, sacks, wood, furniture, and the like.

### EXAMPLES

### Materials and Methods

### Plants:

Arabidopsis thaliana (L.) Heynh. ecotype Columbia
Nicotiana tabacum L. cv samsun NN
Kenaf—Hibiscus cannabinus L. cv Tainung 2
Agrobacterium tumefaciens strain:
GV3101—Used for transformation of kenaf, Arabidopsis, and tobacco (Hellens et al. (2000) Trends Plant Sci 5: 446-451). Antibiotic resistance: Rifampicin and Gentamycin.

### Bacterial Growth Media

Growth media for bacteria was prepared as previously described (Sambrook et al. (1989) Molecular Cloning, A Laboratory Manual. Cold Spring Harbor: Cold Spring Harbor Laboratory Press). For solid media 1.5% w/v of agar was added. Antibiotics were added to a final concentration of: Ampicillin 100 μg/ml, Kanamycin 50 μg/ml for bacteria and 100-250 μg/ml for plants, Spectinomycin 100 μg/ml, Rifampicin 100 μg/ml, and Gentamycin 100 μg/ml.

### Plant Growth Media

### Murashige & Skoog—MS

4.4 g MS #M0222 (Duchefa Biochemic B.V., The Netherlands) medium and 15 g sucrose per liter. pH was adjusted to 5.8 with 1N KOH. For solid media 0.6% w/v Plant Agar #P1001 was added (Duchefa Biochemic B.V., The Netherlands). Antibiotics (if required) were added to a final concentration of: Kanamycin 100-250 μg/ml Hygromycin 15 μg/ml, and G418 15 μg/ml.

### Primers

| Primer | Sequence (5′→3′) | Intended purpose |
|---|---|---|
| CaMV 35S pro end | CAAGACCCTTCCTCTATATAAG | Identifying CAMV 35S |
| | SEQ ID NO: 5 | promoter presence |
| NOS terminator | TTATCCTAGTTTGCGCGCTA | Identifying NOS |
| reverse | SEQ ID NO: 6 | terminator presence |
| M13-20 | TGTAAAACGACGGCCAGT | Universal primers |
| | SEQ ID NO: 7 | |
| M13-21 | AACAGCTATGACCATGATTACG | |
| | SEQ ID NO: 8 | |
| GMO35S REV | GCTCCTACAAATGCCATCA | Identifying CAMV 35S |
| | SEQ ID NO: 9 | promoter positive |
| GMO35S FOR | GATAGTGGGATTGTGCGTCA | transgenic plants |
| | SEQ ID NO: 10 | |
| AT-GA20Hope5′ | ATGGATCCTATGGCCGTAAGTTTCGTA ACAAC | pBinPlus cloning |
| | SEQ ID NO: 11 | (BamHI and EcoRI) |
| AT-GA20Hope3′ | ACGAATTCGGTTAGATGGGTTTGGTGAGCCAA | |
| | TCTG | |
| | SEQ ID NO: 12 | |
| AT-GA20Over5′ | GGTCTAGAATGGCCGTAAGTTTCGTAACAAC | pBIN-GFP cloning (XbaI) |
| | SEQ ID NO: 13 | |
| AT-GA20Over3′ | CCTCTAGAGATGGGTTTGGTGAGCC | |
| −stop | SEQ ID NO: 14 | |
| AT-gA20Over3′ | CCTCTAGATTAGATGGGTTTGGTGAGCC | |
| +stop | SEQ ID NO: 15 | |
| ATGA2C1aKpn | CCATCGATGGTACCCCTGAAAGTTCCCGGGCT | pKannibal cloning |
| TTTG | SEQ ID NO: 16 | |
| ATGA2BamHI- | CGGGATCCCGGAATTCCGCAATGGCGGTATTG | |
| EcoRI | TCTAAACC | |
| | SEQ ID NO: 17 | |
| GA2TABACsense | ACTCGAGAGATTGGATTTGGTGAGC | pKannibal cloning |
| 5′ | SEQ ID NO: 18 | |
| GA2TABACanti | AATCGATAACCTGCAATGAGTCACC | |
| 5′ | SEQ ID NO: 19 | |
| GA2TABACsense | AGGTACCAACCTGCAATGAGTCACC | |
| 3′ | SEQ ID NO: 20 | |
| GA2TABACanti | GTCTAGAAGATTGGATTTGGTGAGC | |
| 3′ | SEQ ID NO: 21 | |
| 5′ kenaf anti | GATCGATGCAGAGTCACTCTGCTCGTCC | pKannibal cloning |
| | SEQ ID NO: 22 | |
| 3′ kenaf sense | CGGTACCCCAGAGTCACTCTGCTCGTCC | |
| | SEQ ID NO: 23 | |
| 5′ kenaf anti | GTCTAGAGGTGATCAACCATGGGGTTCCC | |
| | SEQ ID NO: 24 | |
| 3′ kenaf sense | CCTCGAGGGTGATCAACCATGGGGTTCCC | |
| | SEQ ID NO: 25 | |
| GA2kenaf nested | GGGCAGCCCAAACCTTATGG | Nested primer for the kenaf |
| | SEQ ID NO: 26 | GA2-Oxidase gene |
| GA2kenaf5′ | ATGGTGTTATTATCAAAACCTGGAATTG | Primers for the kenaf GA2- |
| | SEQ ID NO: 27 | Oxidase gene |
| GA2kenaf3′ | GCCTAAAAGATAGAGAGATTATGAGGCAGC | |
| | SEQ ID NO: 28 | |
| Inverse1 kenaf | CCATGGGAACCCCATGGTTGATCAC | Primers for inverse PCR used |
| GA2 | SEQ ID NO: 29 | for the kenaf GA2-Oxidase |
| Inverse2 kenaf | GCGGCAAAGAGAAGAACTCGGTGGC | isolation |
| GA2 | SEQ ID NO: 30 | |
| Inverse3 kenaf | CAGGCTGAACCATTACCCTCCATGCCC | |
| GA2 | SEQ ID NO: 31 | |
| Inverse4 kenaf | CGACAATGTGATTGGATTTGGGGAGC | |
| GA2 | SEQ ID NO: 32 | |
| KenafDeg5′ | TGYGARGARTTYGGYTTYTTYAARG | Degenaerate primers for kenaf |
| | SEQ ID NO: 33 | GA2-Oxidase isolation |
| KenafDeg3′ | GGRTCDGTRTGYTCNCGRAANCC | 5′ has 256 permutations |
| | SEQ ID NO: 34 | 3′ has 512 permutations |

### Molecular Procedures

Standard DNA molecular techniques were performed according to Sambrook et al., ibid, and Ausubel et al. (Protocols in Molecular Biology. (1987) New York, N.Y.: John Wiley and Sons).

### Plasmid DNA Production

Plasmid DNA for routine manipulations and sequencing was extracted according to the Alkaline Lysis Miniprep (Sambrook et al., ibid).

### Polymerase Chain Reaction (PCR)

PCR was used for gene amplification, gene detection and cloning. Unless otherwise mentioned, reaction conditions were as follows: 5-100 ng template DNA, 0.05 mM dNTPs, 7.5 μM of each primer, 10 mM Tris pH 8.8, 50 mM KCl, 0.08% Nonidet P40, 1.5 mM MgCl₂, 0.1 mg/ml BSA and 0.125-0.05 μl Taq DNA Polymerase in a final 25 μl volume. Reaction conditions were: 94° C. for 10 minutes, followed by 30 cycles of 94° C. for 1 minute, 54°-62° C. for 1 minute and 72° C. for 3 minutes and a final step of 72° C. for 10 minutes.

### Sequencing and Sequence Analysis

DNA sequencing was carried out by the dideoxynucleotide chain termination method of Sanger (Sanger F. (1981) Science 214: 1205-1210) using a fluorescent DNA automatic sequencer. Homology searches of the database were performed using the Basic Local Alignment Search Tool of the National Center for Biotechnology Information. Nucleotide and amino-acid sequences were analyzed using Clustal W.

### Plant DNA Analysis

### Micro DNA Isolation

DNA extraction buffer: 0.35M Sorbitol, 0.1M Tris-HCl pH-7.5, 5 mM EDTA. 0.02M Na Bisulfite was added before use.

Nuclei lysis buffer: 0.2M Tris pH-7.5, 50 mM EDTA, 2M NaCl, 2% CTAB.

Extraction buffer mix—1 volume DNA Extraction buffer, 1 volume Nuclei lysis buffer, 0.4 volume 5% Sarcosyl (w/v in ddH₂O).

Hundred mg of fresh leaves, from the apical meristem region, were frozen and ground in liquid nitrogen using a wooden toothpick. Ground tissues were then homogenized with 400 μl extraction buffer mix and vortexed for 1 minute. Homogenized mixtures were then incubated for 40 minutes in 65° C. Lysates were extracted once with 1:1 volume:volume of chloroform. Tubes were vortexed for 5 minutes followed by centrifugation for 5 minutes at 15,000 g in an Eppendorff centrifuge. Two-thirds volume of isopropanol and 0.1 volumes of 3M NaOAC pH 6 were added to the aqueous phase DNA precipitation. Tubes were incubated for 30 minutes in −20° C. and then centrifuged at 20,000 g for 30 minutes at 4° C. DNA pellets were washed once with 70% ethanol and dissolved in 100 μl ddH₂O.

### Southern Blot Analysis

Restriction digests, electrophoresis on agarose gel, Southern blots, hybridization and autoradiography were basically performed as described by Bernatzky and Tanksley (Plant Mol. Biol. Rep. 4: 37-41, 1986) and Sambrook et al. ibid.

### Primer Labeling Mix:

TM—0.25M Tris pH 8, 0.025M MgCl₂, 0.005M DTT (stored at 4° C.)
OL—90 Units/ml of Hexadeoxynucleotide (Boehringer Mannheim) in TE (stored at −20° C.)
DTM—0.1 mM dATP, dGTP and dTTP in TM (stored at −20° C.)
LS—500 μl HEPES 1M pH 6.6, 500 μl DTM, 140 μl OL (stored at −80° C.)

Forty μg of genomic DNA were digested with the desired restriction enzyme, using buffers recommended by the supplier in a 150 μl reaction volume with the addition of spermidine to a final concentration of 4 mM and Rnase A to a final concentration of 100 μg/ml. Digested DNA fragments were separated on a 1% agarose gel prestained with ethidium bromide 0.5 μg/ml using the NEB buffer (0.1M Tris base, 10 mM EDTA pH-8.1, 126 mM NaAcetate). After electrophoresis, gels were photographed under U.V. light. The DNA was nicked by UV irradiation (254 nm) for 3 minutes prior to capillary transfer. The DNA embedded agarose gel was incubated for 1.5 hours in a denaturation solution (1M NaCl, 0.5M NaOH) and then washed 3 times in sterile ddH₂O. The agarose gel was then incubated for another 1.5 hours in a neutralization solution (1.5M NaCl, 0.5M Tris pH 7) and then washed two times in ddH₂O. DNA was blotted onto Hybond N⁺ (Amersham-Biosciences, Sweden) membranes using 20×SSC for 20 hours. Blots were washed in 2×SSC and dried. Probes were labeled with P³² dCTP using the following protocol:

About fifty ng of DNA were boiled with 18 μl ddH₂O for 5 minutes and immediately cooled on ice prior to the addition of 11 μl LS, 5 μl BSA (1 mg/ml), 5 μl dCTP and 1 μl Klenow fragment DNA polymerase (total volume 40 μl). The labeling mixture was incubated for 1 hour at 37° C. and unincorporated nucleotides were removed using ProbeQuant G50 spin micro columns (Amersham).

### Prehybridization and Hybridization

Prehybridization and hybridization were performed in plastic/glass tubes in an orbital shaker incubator at 25 rpm at 65° C. Membranes were prehybridized in 45 ml hybridization buffer containing 0.263M Na₂HPO₄, 7% SDS, 1 mM EDTA, 1% BSA for two hours (20 ml of prehybridization buffer is sufficient for one 20 cm×10 cm blot). The probe was denatured for 10 minutes in boiling water before added to 30 ml hybridization buffer. The hybridization was carried out overnight in an orbital shaker at 65° C. Membranes were then washed once in 0.263M Na₂HPO₄, 1% SDS for 20 minutes in 65° C., followed by 1 wash of 20 minutes with 2×SSC, 0.1% SDS and a third wash of 20 minutes with 1×SSC, 0.1% SDS. Membranes were then lightly dried, plastic wrapped and exposed to Kodak Bio max-MS X-ray film for 24-72 hours using Kodak intensifier screen at −80° C.

### Plant RNA Analysis

### Miniprep RNA Isolation

Leaves or seeds were ground with a mortar and pestle under liquid nitrogen. Up to 150 mg were used for total RNA isolation using SV total RNA isolation kit #23200 (Promega, USA) according to the manufacturer's protocol.

### Construction of Silencing Plasmids

The pKannibal vector (Wesley et al. Plant J. 27: 581-590, 2001) was used for silencing GA 2-Oxidases from Arabidopsis, tobacco and kenaf. Two complementing fragments from each plant were cloned PCR using Kannibal primers. Sense fragments were cloned with KpnI and XhoI, antisense fragments were cloned with ClaI and Xba1. The sense and antisense orientations were cloned into a pKannibal vector. The pKannibal containing the sense and antisense fragments was digested with NotI and then sub-cloned into the binary vector pArt27 (Gleave, A. P. Plant Mol Biol 20: 1203-1207, 1992). The resulting constructs were introduced by electroporation into Agrobacterium tumefaciens GV3101.

### Construction of Over-Expression Plasmids

For the over-expression assays, the genomic Arabidopsis GA20-Oxidase gene was cloned separately into pBinPlus and pNoga (the latter is a variant of pBin19+ containing GFP gene) between the 35SΩ promoter containing the translation enhancer signal and the Nos terminator plus N terminal Myc and C terminal GFP tag, respectively. The resulting constructs were electroporated into Agrobacterium tumefaciens GV3101. These Agrobacterium strains were used to transform the Arabidopsis, tobacco and kenaf plants.

### Plant Transformations

### Arabidopsis Stable Transformations

Transformation was performed by the floral dip method (Clough et al., Plant Journal 16: 735-743, 1998). Briefly: plants were grown until flowering. On the day of transformation the siliques and flowers were clipped. The Agrobacterium culture carrying the binary vector was re-suspended to OD₆₀₀=0.5 in 5% Sucrose solution. Before dipping Silweet L-77 was added to a concentration of 0.05% and mixed well. The plants' aboveground parts were dipped in the Agro solution for 2-3 seconds, with gentle agitation. The dipped plants were placed in a covered tray for 24 h to maintain humidity in the growing room without exposure to extensive light. Plants were grown normally until seeds became mature. The dry seeds were harvested and transformants were selected on MS plates containing 50 μg/ml kanamycin. Putative transformants were transferred to soil.

### Tobacco Stable Transformation

Nicotiana tabacum cv samsun NN plants were transformed as previously described (Horsh et al. Cold Spring Harb Symp Quant Biol 50: 433-437, 1985). Briefly, tobacco plants were grown under sterile conditions in Magenta boxes containing solidified (0.6% Plant Agar, Duchefa, The Netherlands) MS medium. Leaves were cut to small rectangles and incisions were made towards the main vein. The leaves were then incubated for 20 minutes in an overnight culture of Agrobacterium tumefaciens containing the desired vector diluted in MS liquid medium to a final OD₆₀) of 0.5. After blotting on sterile paper the leaves were co-cultivated for 2-3 days on a solidified MS medium supplemented with 2 mg/ml kinetin and 0.8 mg/ml IAA (Sigma). Then the leaves were transferred to solidified MS medium containing 2 mg/ml kinetin, 0.8 mg/ml IAA, 400 mg/liter claforan and 100 mg/ml kanamycin. Calli were transferred to fresh medium every two weeks until shoot regeneration. Regenerating shoots were then transferred to a solidified MS medium supplemented with 100 mg/liter kanamycin and 400 mg/liter claform. Regenerated shoots that rooted in the presence of 100 mg/liter kanamycin were potted and transferred to a 25° C. growth chamber. Positive kanamycin resistant plants were examined for the presence of the inserted gene by either PCR or Southern blot analysis.

### Transient Expression Assays

Binary vector clones were introduced by electroporation into Agrobacterium tumefaciens strain GV3101. Agrobacterium cells were grown in LB medium overnight at 28° C., diluted into VIR induction medium (50 mM MES pH 5.6, 0.5% (w/v) glucose, 1.7 mM NaH₂PO₄, 20 mM NH₄C1, 1.2 mM MgSO₄, 2 mM KCl, 17 μM FeSO₄, 70W CaCl₂ and 200 μM acetosyringone) and grown for 6 additional hours until OD₆₀₀ reached 0.4-0.5. The Agrobacterium cultures were then diluted to a final OD₆₀₀ of 0.2 and the suspensions was injected with a needless syringe into leaves of tobacco and kenaf plants. Leaves were observed under a confocal microscope for GFP expression.

### Tissue Preparation and Microscopy

One to three mm thick sections of stem tissues were hand-cut. The sections were stained at room temperature with a 0.4% solution of lacmoid (PolyScience, Niles, Ill.) in 90% lactic acid for about 2 sec, and then rinsed in tap water until the residual color was washed away. Subsequently, the sections were transferred to 50% sodium lactate for microscopic analysis under transmitted white light (Aloni and Barnett, Planta 198: 595-603, 1996).

### Kenaf GA 2-Oxidase and Partial Tomato GA 2-Oxidase Gene Isolation

### Degenerate Primers

Four different plant GA 2-Oxidase amino acid sequences were aligned (NCBI accession numbers: Q8LEA2, Q9XFR9, BAD17856.1, and AAQ93035.1). Highly conserved regions were identified using Clustal W. The longest most conserved region was reverse translated with MacVector software (Accelrys Software Inc.). Permutations were reduced according to plant codon usage (Murray E. et al., 1999). Resulting degenerate primer sequences were:

| | |
|---|---|
| 5′ Forward: | 5′-TGYGARGARTTYGGYTTYTTYAARG-3′ |
| 3′ Reverse: | 5′-GGRTCDGTRTGYTCNCGRAANCC-3′ |

### Inverse PCR

PCR on kenaf and tomato extracted genomic DNAs was performed using the degenerate primers (listed hereinabove). The resulting amplicons were cloned into the pTZ57R/T plasmid and sequenced. According to this sequence 4 primers were designed (i.e. Inverse 1-4, see the list of primers herein above) to amplify the genomic DNA flanking the PCR product. Two were outer primers and two for a subsequent PCR. DNA template for inverse PCR was prepared by treatment of 10 μg of kenaf genomic DNA with 20 U of MseI at 37° C. for 3 h, followed by ethanol precipitation. MseI restriction endonuclease was chosen because its recognition sequence was not found within the coding sequence of the amplified fragment. One microgram of MseI-treated DNA was recircularized with T4DNAligase (4 U) at 4° C. for 12 h followed by ethanol precipitation. A PCR reaction was then executed on 100 ng of self-ligated plant genomic DNA using the inverse primers. The PCR product was cloned into the pTZ57R/T plasmid and sequenced. NCBI database were then searched for homologous sequences to the kenaf isolated gene.

### Example 1

### Transgenic Arabidopsis Lines

Transformation of Arabidopsis Plants with a GA 20-Oxidase Gene Driven by an Enhanced 35S Promoter

Primers were designed based on the reported Arabidopsis GA 20-Oxidase sequence (Xu et al. Proc. Natl. Acad. Sci. USA 92: 6640-6644, 1995). These primers were used to isolate the GA 20-Oxidase genomic sequence. The sequence of the PCR-amplified gene was similar to that of GA5 (accession no. ATU20873). Blast analysis between the cloned gene and the reported gene has shown a couple of discrepancies, however no gaps or stop codons have been found. These base-pair changes were unlikely to be mutations resulting from the PCR reaction since they were adjacent to one another and they appeared in a very high frequency, not concurrent to Taq polymerase error frequency. The GA5 sequence was isolated from the Columbia ecotype and introduced into the binary Ti plasmid, pBin19Plus. The GA 20-Oxidase gene was cloned under the regulation of the 35S promoter and ligated to a Myc epitope at its N terminus through the pLola mediation vector. Arabidopsis plants were transformed using Agrobacterium tumefaciens strain GV3101. Transgenic T1 lines were selected by growth on kanamycin-containing medium. The T1 plant lines that were randomly selected were considerably taller than the wt control plants.

Transformation of Arabidopsis Plants with a GA 2-Oxidase Gene Suppression Construct

Primers were designed, based on the NCBI submitted Arabidopsis GA 2-Oxidase sequence (NCBI accession no. NM_{—}106491), to isolate a 373 by fragment (SEQ ID NO:35), the sequence of which is as follows:

CAATGGCGGTATTGTCTAAACCGGTAGCAATACCAAAATCCGGGTTCT
CTCTAATCCCGGTTATAGATATGTCTGACCCAGAATCCAAACATGCCC
TCGTGAAAGCATGCGAAGACTTCGGCTTCTTCAAGGTGATCAACCATG
GCGTTTCCGCAGAGCTAGTCTCTGTTTTAGAACACGAGACCGTCGATT
TCTTCTCGTTGCCCAAGTCAGAGAAAACCCAAGTCGCAGGTTATCCCT
TCGGATACGGGAACAGTAAGATTGGTCGGAATGGTGACGTGGGTTGGG
TTGAGTACTTGTTGATGAACGCTAATCATGATTCCGGTTCGGGTCCAC
TATTTCCAAGTCTTCTCAAAAGCCCGGGAACTTTCAG

The fragment was cloned into the pKannibal vector in both sense and anti-sense orientations, flanking the Pdk intron (Wesley et al., Plant J. 27: 581-590, 2001). This vector was sub-cloned into the binary vector pArt27 (Gleave, Plant Mol Biol 20: 1203-1207, 1992). The vector was electroporated into Agrobacterium tumefaciens GV3101 strain used for plant transformation. Transgenic T1 lines were selected by growth on kanamycin-containing medium.

The complementary sequence of SEQ ID NO:35 is as follows (SEQ ID NO:39):

CTGAAAGTTCCCGGGCTTTTGAGAAGACTTGGAAATAGTGGACCCGAA
CCGGAATCATGATTAGCGTTCATCAACAAGTACTCAACCCAACCCACG
TCACCATTCCGACCAATCTTACTGTTCCCGTATCCGAAGGGATAACCT
GCGACTTGGGTTTTCTCTGACTTGGGCAACGAGAAGAAATCGACGGTC
TCGTGTTCTAAAACAGAGACTAGCTCTGCGGAAACGCCATGGTTGATC
ACCTTGAAGAAGCCGAAGTCTTCGCATGCTTTCACGAGGGCATGTTTG
GATTCTGGGTCAGACATATCTATAACCGGGATTAGAGAGAACCCGGAT
TTTGGTATTGCTACCGGTTTAGACAATACCGCCATTG

### Phenotypic Characterization of Transgenic Arabidopsis Lines

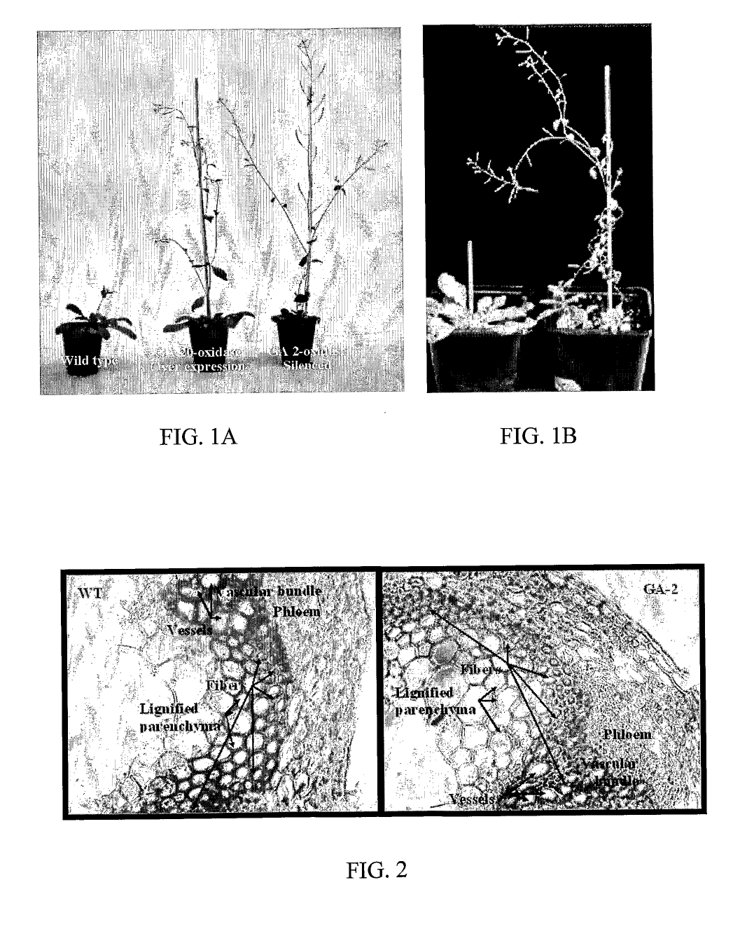

T1 lines of both transformations, GA 20-Oxidase over expression and GA 2-Oxidase silencing, were planted, and leaf samples were collected. Genomic DNA was extracted and tested for the presence of the transgene by PCR. Lines positive to the transgene were planted along side the control wild type (wt) plants. During the plants' growth the anatomical characteristics were analyzed and compared to the wt control plants. GA 20-Oxidase over expressing plants showed similar phenotypes to the GA 2-Oxidase silenced plants. The transgenic plants grew taller and flowered faster under long day conditions (FIG. 1A). Significant variance in the number of internodes constituting the inflorescence axis was not detected. Therefore, the increase in plant growth can be attributed to internode lengthening. Under short day conditions wt plants grew typical large rosettes while the transgenic lines grew inflorescence axis typical to long day conditions (FIG. 1B). Stem cross sections were stained with lacmoid (identifying lignin in the fiber cells). Fibers in the silenced GA 2-oxidase transgenic lines were stained light blue in comparison to the dark blue wt fibers, likely indicating a reduction in lignin accumulation in the cell walls (FIG. 2). Furthermore, a large increase in xylem fiber count and density was detected in the transgenic lines.

### Example 2

### Transgenic Tobacco Lines

### Heterologous Over-Expression of Arabidopsis GA 20-Oxidase Gene in Tobacco Plants

Nicotiana tabacum plants were transformed with the same Agrobacterium tumefaciens that were used to insert the GA20-Oxidase gene into Arabidopsis. The Arabidopsis GA 20-Oxidase gene was cloned into the binary vector pNOGA between the 35S-Ω promoter containing the translation enhancer signal and the GFP gene (green fluorescence protein) followed by the NOS Terminator. Transgenic T1 lines were selected by growth on kanamycin-containing medium. Western blot analysis confirmed protein expression.

Transformation of Tobacco Plants with a GA 2-Oxidase Gene Suppression Construct

Primers were designed, based on the NCBI submitted tobacco GA 2-Oxidase sequence (accession no. AB125232 and AB125233), to isolate a 155 by silencing fragment (SEQ ID NO:36), the sequence of which is as follows:

GATTGGATTTGGAGAGCATACTGACCCACAAATCATATCAGTATTGAG
ATCCAACAACACTTCCGGACTTCAAATATTACTCAAAAATGGCCACTG
GATTTCTGTCCCACCTGATCCAAATTCTTTCTTCATTAATGTTGGTGA
TTCATTGCAGG

The sequence that was chosen complements the two NCBI reported tobacco GA 2-Oxidase family genes. The fragment was cloned into the pKannibal vector in both sense and anti-sense orientations, flanking the Pdk intron. This vector was sub-cloned into the binary vector pART27. The vector was electroporated into Agrobacterium tumefaciens GV3101 strain used for plant transformation. Transgenic T1 lines were selected by growth on kanamycin-containing medium.

The complementary sequence of SEQ ID NO:36 is as follows (SEQ ID NO:40):

CCTGCAATGAATCACCAACATTAATGAAGAAAGAATTTGGATCAGGTG
GGACAGAAATCCAGTGGCCATTTTTGAGTAATATTTGAAGTCCGGAAG
TGTTGTTGGATCTCAATACTGATATGATTTGTGGGTCAGTATGCTCTC
CAAATCCAATC

### Phenotypic Characterization of Transgenic Tobacco Lines

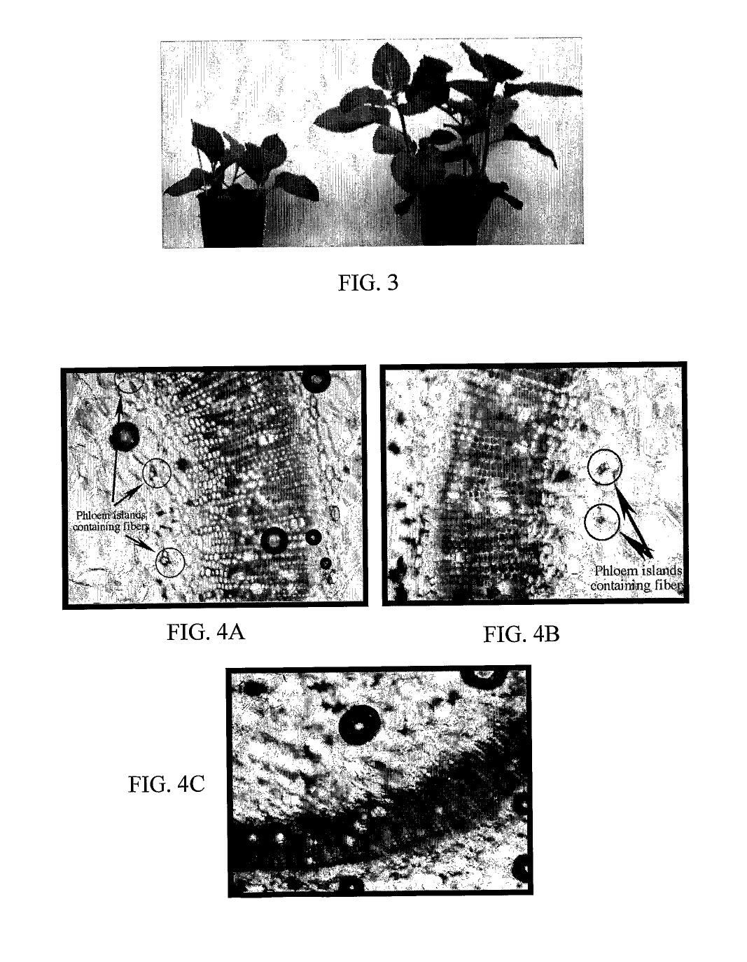

T1 lines of heterologous GA 20-Oxidase over expression were planted. Leaves were cut and observed under a confocal microscope for GFP luminescence. Lines expressing the GFP were planted along side wt plants. After about 2.5 months, anatomical measurements were taken from internodes along the stem. Transformed lines were taller than the control (FIG. 3). A significant increase in the number of internodes was detected between three transgenic lines and the wt, in contrary to the phenotype in Arabidopsis. However, as in Arabidopsis, the transformation resulted in longer internode lengths. The Internode length difference was more prominent in the longest internodes. Stem cross-sections were prepared and observed under a light microscope following lacmoid staining. Transformed plants produced dramatically more inner phloem fibers (FIG. 4) and higher overall fiber count (Table 2) compared to the wt control. In agreement with the difference in internode length, a detectably higher number of developmental stage internodes were identified in the transgenic plants given they did not exhibit fiber initiation that was observed in the same internodes of the wt control plants, thereby indicating that GA 20-Oxidase over-expression accelerates plant growth. Reduction in lignin found in Arabidopsis was not prominent in tobacco transgenic plants.

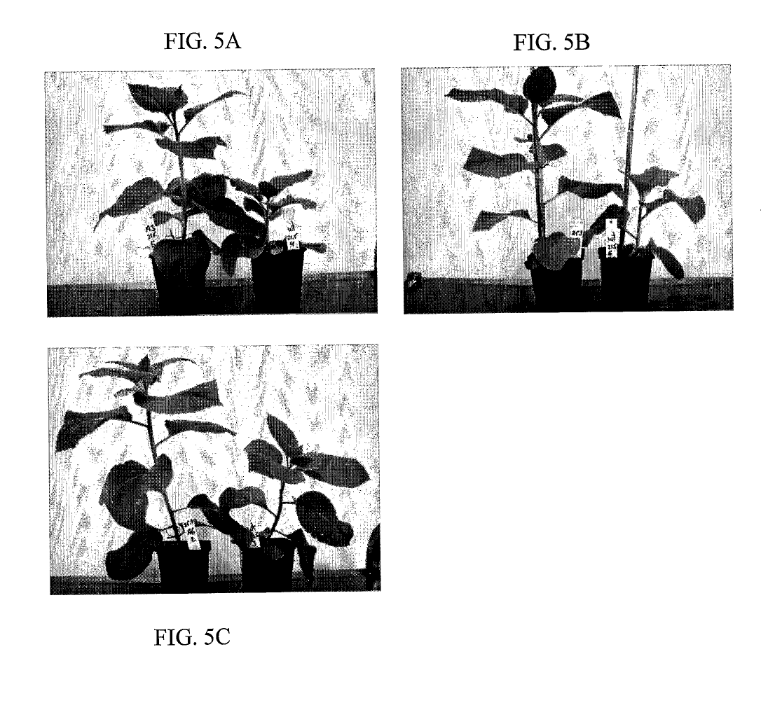

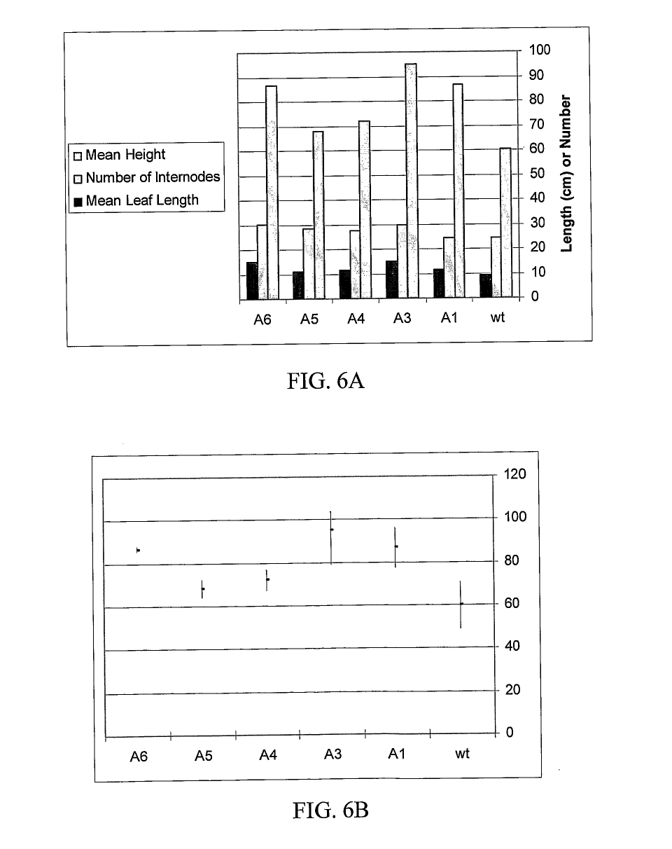

GA 2-Oxidase silenced T1 lines were planted along wt controls. Anatomical characteristics have been measured. The number of internodes did not differ significantly between the two groups although the four tallest lines were 20%-60% taller (FIGS. 5 and 6). Leaf size has also been impacted by the silencing. Transgenic leaves were longer and resided in higher internodes then in the wt plants.

### Example 3

### Isolation of kenaf and Tomato Silencing Fragments

Degenerate primers for GA 2-Oxidase were designed on the basis of NCBI submitted sequences from a range of species as described in Materials and Methods herein above. These primers were used for PCR analysis on genomic DNA that was extracted from young leaves of kenaf and tomato. Low stringency PCR was programmed using a hybridization temperature of 54° C. The amplified kenaf fragment was 473 by in length and did not appear to include any introns (see herein below). Therefore, it was used to construct a silencing vector via pKANNIBAL as described for Arabidopsis and tobacco. The amplified tomato fragment was 488 by long (see herein below). Both sequences were confirmed as GA 2-Oxidase fragments by their translation and BLAST analysis at the amino acid level.

The kenaf amplified fragment sequence (SEQ ID NO: 4) was as follows:

GTGAGGAGTTTGGTTTCTTCAAAGTGATCAACCATGGGGTTCCCATGG
AATTCATTTCCAGGCTTGAATCTGAAGCCACCGAGTTCTTCTCTTTGC
CGCTTTCTGAGAAGGAGAAAACAGGGCAGCCCAAACCTTATGGATATG
GCAATAAAAGGATCGGTCCCAATGGTGATGTTGGTTGGGTTGAATATC
TTCTCCTCATGACCAACCAAGACCCCAACCTCGCTACTGAAAACCCAG
AAAGTTTCAGGGTTGCTTTGAATAATTATATGGCGGCGGTGAAGAAGA
TGGCGTGCGAGATACTGGAAATGGTGGCGGATGGGCTGAAGATTCAAC
CGAGGAATGTGTTGAGCAAGCTGATGATGGACGAGCAGAGTGACTCTG
TTTTCAGGCTGAACCATTACCCTCCATGCCCAGATGTTGTTCAATCTC
TCAACGACAATGTGATTGGATTTGGGGAGCACACAGACCCA

The complementary sequence of SEQ ID NO:4 is as follows (SEQ ID NO:38):

TGGGTCTGTGTGCTCCCCAAATCCAATCACATTGTCGTTGAGAGATTG
AACAACATCTGGGCATGGAGGGTAATGGTTCAGCCTGAAAACAGAGTC
ACTCTGCTCGTCCATCATCAGCTTGCTCAACACATTCCTCGGTTGAAT
CTTCAGCCCATCCGCCACCATTTCCAGTATCTCGCACGCCATCTTCTT
CACCGCCGCCATATAATTATTCAAAGCAACCCTGAAACTTTCTGGGTT
TTCAGTAGCGAGGTTGGGGTCTTGGTTGGTCATGAGGAGAAGATATTC
AACCCAACCAACATCACCATTGGGACCGATCCTTTTATTGCCATATCC
ATAAGGTTTGGGCTGCCCTGTTTTCTCCTTCTCAGAAAGCGGCAAAGA
GAAGAACTCGGTGGCTTCAGATTCAAGCCTGGAAATGAATTCCATGGG
AACCCCATGGTTGATCACTTTGAAGAAACCAAACTCCTCAC

The tomato amplified fragment sequence (SEQ ID NO:37) was as follows:

TTGCGAGGAATTTGGTTTTTTTAAAGTCGTAAACCATGACGTTCCTAT
AGAATTCATAAGTAAACTCGAATCCGAAGCCATCAAATTCTTCTCCTC
TCCCCTCTCTGAGAAGCTAAAAGCAGGGCCTGCTGACCCTTTTGGCTA
TGGCAATAAACAAATCGGACAAAGTGGCGATATCGGTTGGGTTGAATA
CATTCTCCTTTCAACAAACTCTGAATTCAATTACCAGAAATTCGCATC
TGTATTAGGTGTCAATCCAGAAAACATTCGGTAAGTCATTAAAAGCAG
AGCAAAACAGAGTGATGTCTTTTAATCTCGATTCTTGATTTTTGTTAT
TGTTATTGNTATTGCAGAGCTGCGGNGAANGATTATGTGGCAGCAGTG
AAGAGAANGTCATGTGAGATTCTTGAAGAAGTTGGCGGAGGGATTAAA
GATTCATCCGNCGAATGTTTTCGGTAAGCTTTTGAAGGATGAAAAGAG
CGACTCTG

The complementary sequence of SEQ ID NO: 37 is as follows (SEQ ID NO:41):

CAGAGTCGCTCTTTTCATCCTTCAAAAGCTTACCGAAAACATTCGNCG
GATGAATCTTTAATCCCTCCGCCAACTTCTTCAAGAATCTCACATGAC
NTTCTCTTCACTGCTGCCACATAATCNTTCNCCGCAGCTCTGCAATAN
CAATAACAATAACAAAAATCAAGAATCGAGATTAAAAGACATCACTCT
GTTTTGCTCTGCTTTTAATGACTTACCGAATGTTTTCTGGATTGACAC
CTAATACAGATGCGAATTTCTGGTAATTGAATTCAGAGTTTGTTGAAA
GGAGAATGTATTCAACCCAACCGATATCGCCACTTTGTCCGATTTGTT
TATTGCCATAGCCAAAAGGGTCAGCAGGCCCTGCTTTTAGCTTCTCAG
AGAGGGGAGAGGAGAAGAATTTGATGGCTTCGGATTCGAGTTTACTTA
TGAATTCTATAGGAACGTCATGGTTTACGACTTTAAAAAAACCAAATT
CCTCGCAA

### Example 4

### The Complete kenaf GA 2-Oxidase Gene Isolation

DNA fragments obtained by PCR using the degenerate primers were used to design primers for inverse PCR followed by a nested PCR as described in Materials and Methods herein above. The nested PCR amplicon was cloned into the pUC57R/T plasmid and sequenced. The sequence was verified by three independent sequences on three independent amplicon embedded plasmids. Hence, the kenaf GA 2-Oxidase complete genomic sequence (SEQ ID NO:3) was identified (FIG. 7).

The gene is 1134 bp long and includes numerous putative splice variants. It has no apparent DNA homology to any known gene, however, there is a 63% homology and 76% similarity to the GA20x2 of Nerium oleander at the amino acid level. The cDNA nucleotide sequence (SEQ ID NO:1) and the amino acid sequence (SEQ ID NO:2) of GA 2-oxidase of Kenaf is shown in FIG. 8.

It will be appreciated by persons skilled in the art that the present invention is not limited by what has been particularly shown and described herein above. Rather the scope of the invention is defined by the claims that follow.

1.-89. (canceled)

90. A double stranded RNA molecule that down regulates expression of GA 2-oxidase gene of Hibiscus cannabinus (kenaf) comprising:
a) a first RNA strand of the double stranded RNA comprising at least 20 contiguous nucleotides having at least 90% sequence identity to an RNA transcribed from a GA 2-oxidase gene of kenaf or a fragment thereof; and
b) a second RNA strand of said double stranded RNA comprising at least 20 contiguous nucleotides having at least 90% sequence identity to a complementary sequence of the RNA transcribed from the GA 2-oxidase gene of kenaf or a fragment thereof,
wherein the first and the second RNA strands are capable of hybridizing to each other to form said double stranded RNA molecule.

91. The double stranded RNA molecule according to claim 90, wherein the RNA transcribed from the GA 2-oxidase gene of kenaf comprises the nucleotide sequence as set forth in SEQ ID NO:1.

92. The double stranded RNA molecule according to claim 90, wherein the fragment of the RNA transcribed from the GA 2-oxidase gene of kenaf comprises the nucleotide sequence as set forth in SEQ ID NO:4.

93. A DNA construct for generating an RNA molecule capable of down regulating expression of a GA 2-oxidase gene of kenaf, the DNA construct comprising a plant promoter operably linked to a polynucleotide sequence encoding an RNA sequence that forms a double stranded RNA, the polynucleotide sequence comprising:
(i) a first nucleotide sequence comprising at least 20 contiguous nucleotides having at least 90% sequence identity to the nucleotide sequence as set forth in SEQ ID NO:1 or a fragment thereof; and
(ii) a second nucleotide sequence comprising at least 20 contiguous nucleotides having at least 90% sequence identity to a complementary sequence of the nucleotide sequence as set forth in SEQ ID NO:1 or a fragment thereof,
wherein the RNA molecule transcribed from the DNA construct down regulates the expression of the GA 2-oxidase gene of kenaf.

94. The DNA construct according to claim 93, wherein the first and second nucleotide sequences each comprises at least 100 nucleotides.

95. The DNA construct according to claim 93, wherein the first nucleotide sequence has at least 90% sequence identity to the nucleotide sequence as set forth in SEQ ID NO:4 or a fragment thereof.

96. The DNA construct according to claim 93, wherein the second nucleotide sequence has at least 90% sequence identity to the nucleotide sequence as set forth in SEQ ID NO:38 or a fragment thereof.

97. A plant cell transformed with a double stranded RNA according to claim 90.

98. The plant cell according to claim 97, wherein the cell is a kenaf plant cell.

99. A plant cell transformed with a DNA construct according to claim 93.

100. The plant cell according to claim 99, wherein the cell is a kenaf plant cell.

101. A transgenic plant comprising at least one cell transformed with a double stranded RNA according to claim 90.

102. The transgenic plant according to claim 101 is a kenaf plant.

103. A transgenic plant comprising at least one cell transformed with a DNA construct according to claim 93.

104. The transgenic plant according to claim 103 is a kenaf plant.

105. A seed of a transgenic plant, wherein the transgenic plant is a kenaf plant according to claim 102.

106. A seed of a transgenic plant, wherein the transgenic plant is a kenaf plant according to claim 104.

107. A stem of a transgenic plant, wherein the transgenic plant is a kenaf plant according to claim 102.

108. A stem of a transgenic plant, wherein the transgenic plant is a kenaf plant according to claim 104.

109. A method for producing a transgenic plant having reduced expression of GA 2-oxidase gene, the method comprises introducing into at least one cell of the plant a double stranded RNA according to claim 90.

110. The method according to claim 109, wherein the transgenic plant is a kenaf plant.

111. A method for producing a transgenic plant having reduced expression of a GA 2-oxidase gene, the method comprises introducing into at least one plant cell a DNA construct comprising a plant promoter operably linked to a polynucleotide sequence encoding an RNA sequence, the polynucleotide sequence comprising:
a first nucleotide sequence comprising at least 20 contiguous nucleotides having at least 90% sequence identity to a nucleic acid molecule encoding a GA 2-oxidase of the plant or a fragment thereof; and
(ii) a second nucleotide sequence comprising at least 20 contiguous nucleotides having at least 90% sequence identity to a complementary sequence of the nucleic acid molecule encoding the GA 2-oxidase of the plant or a fragment thereof,
wherein the RNA molecule transcribed from the DNA construct down regulates expression of said GA 2-oxidase gene.

112. The method according to claim 111, wherein the plant is selected from the group consisting of angiospermae families and gymnospermae families.

113. The method according to claim 112, wherein the plant is a kenaf plant.

114. A method for increasing the fiber crop of a plant comprising introducing into at least one plant cells a DNA construct comprising a plant promoter operably linked to a polynucleotide sequence encoding an RNA sequence, the polynucleotide sequence comprising:
(i) a first nucleotide sequence comprising at least 20 contiguous nucleotides having at least 90% sequence identity to a nucleic acid molecule encoding a GA 2-oxidase of the plant or a fragment thereof; and
(ii) a second nucleotide sequence comprising at least 20 contiguous nucleotides having at least 90% sequence identity to a complementary sequence of the nucleic acid molecule encoding the GA 2-oxidase of said plant or a fragment thereof,
wherein the RNA molecule transcribed from the DNA construct down regulates expression of the GA 2-oxidase gene.

115. The method according to claim 114, wherein the plant is selected from the group consisting of angiospermae families and gymnospermae families.

116. The method according to claim 115, wherein the plant is a kenaf plant.

117. An isolated nucleic acid molecule comprising the nucleotide sequence as set forth in SEQ ID NO:1 encoding a gibberellin 2-oxidase (GA 2-oxidase) of Hibiscus cannabinus (kenaf), or a complementary strand or homologous sequence thereto, wherein the homologous sequence has sequence identity of at least 80% to SEQ ID NO:1.
